(19) United States
(12) Patent Application Publication
(10) Pub. No.: US 2009/0117561 A1
(43) Pub. Date: May 7, 2009

(54) DIFFERENTIAL EXPRESSION GENE PROFILES AND APPLICATIONS IN MOLECULAR STAGING OF HUMAN GASTRIC CANCER

(76) Inventors: Youyong Lu, Beijing (CN); Ruifang Guo, Beijing (CN); Shizhu Zang, Beijing (CN); Liang Zhang, Beijing (CN); Yonghong Ren, Beijing (CN); Yonglong Zhuang, Beijing (CN); Jing Cheng, Beijing (CN)

Correspondence Address: MORRISON & FOERSTER LLP, 12531 HIGH BLUFF DRIVE, SUITE 100, SAN DIEGO, CA, 92130-2040, US

(21) Appl. No.: 11/997,313

(22) PCT Filed: Oct. 13, 2005
(86) PCT No.: PCT/CN2005/001684

§ 371 Date: Aug. 1, 2008

(30) Foreign Application Priority Data
Aug. 2, 2005 (CN) 200510088893.0

(51) Int. Cl. C12Q 1/68

(52) U.S. Cl. 435/6

(57) ABSTRACT

The invention provides methods for detecting differential gene expression in intestinal gastric tissue in a mammal by comparing the expression of specific genes in an intestinal gastric tissue suspected of being cancerous with that of the corresponding adjacent intestinal gastric tissue or a normal gastric mucosa tissue. The methods can be used in diagnosing or monitoring the progression of intestinal gastric cancer and determining the levels of differentiation of intestinal gastric cancer Systems and kits for methods of the invention are also provided.

### TECHNICAL FIELD

This application is in the field of gastric cancer. In particular, this invention relates to methods and compositions of diagnosing and monitoring progression of gastric cancer.

### BACKGROUND OF THE INVENTION

Current methods for diagnosing and treating tumors are based primarily on tissue biopsy or other anatomy-based methods. Patients are diagnosed only after space-occupying lesions and clinical symptoms become apparent. Moreover, the diagnoses mainly depend on image analyses and pathology studies. By that time, most of the patients have already developed middle to late-stage cancer, which is difficult to be treated effectively. On the other hand, the lack of effective clinical method of monitoring tumor progression, particularly the lack of an objective standard for selecting a suitable therapeutic regime, often led to improper treatment or even excessive treatment. Therefore, one goal of tumor diagnosis and treatment of tumor is to establish and develop molecular marker profiles that can be used in detecting development and progression of tumors, identifying people who are at risk of developing tumors, classifying tumors, and/or tumor prognosis.

Tumors are complex diseases involving cumulative changes of multiple genes. In the past 30 years, people have obtained a number of tumor-related genes and proteins by cloning and characterizing genes individually. The biological functions of these genes and proteins encoded by these genes were mostly studied by using tumor cell lines. Tumor cell lines are of limited value because they do not accurately reflect gene and protein metabolisms in the human body.

With the development of molecular cell biology and bioinformatics, genes and proteins can now be studied systematically. These studies provide extensive knowledge for understanding the nature of cancer biology, which in turn will lead to effective methods for treating and preventing cancer. For example, due to their high throughput characteristics, gene chips have become effective means for obtaining gene expression profiles of tumor tissues and for classifying tumors.

All references cited herein, including patent applications and publications, are incorporated by reference in their entirety.

### BRIEF SUMMARY OF THE INVENTION

The invention provides compositions, methods, and kits for detection of differential gene expression for diagnosis and monitoring progression of intestinal gastric cancer.

In one aspect, the invention provides a method diagnosing intestinal gastric cancer in a mammal, comprising: (a) detecting levels of expression of at least two genes shown in FIG. 1 in an intestinal gastric tissue of the mammal, wherein the tissue is suspected of being cancerous; (b) detecting levels of expression of the genes in an intestinal gastric tissue adjacent to the tissue suspected of being cancerous of the mammal; and (c) comparing the levels of expression of the genes in the tissue suspected of being cancerous to the levels of expression in the adjacent tissue; wherein substantial variance of the levels of expression of the genes between the tissue suspected of being cancerous and the adjacent tissue is indicative of presence of intestinal gastric cancer in the mammal.

In some embodiments, the levels of expression of at least 5, at least 10, at least 20, at least 30, at least 40, at least 50, at least 60, at least 70, at least 80, at least 90, or at least 100 genes in FIG. 1 are detected. In some embodiments, the levels of expression of all the genes in FIG. 1 are detected, wherein substantially increased expression of at least one of genes 72-102 in FIG. 1 and substantially decreased expression of at least one of genes 1-71 in FIG. 1 in the tissue suspected of being cancerous as compared to the adjacent tissue are indicative of presence of intestinal gastric cancer in the mammal. In some embodiments, the levels of expression of all the genes in FIG. 1 are detected, wherein substantially decreased expression of at least 2, at least 5, at least 10, at least 20, at least 30, at least 40, at least 42, at least 50, at least 60, at least 70, or 71 of genes 1-71 in FIG. 1 and/or substantially increased expression of at least 2, at least 5, at least 10, at least 18, at least 20, or at least 30 of genes 72-102 in FIG. 1 in the tissue suspected of being cancerous as compared to the adjacent tissue are indicative of presence of intestinal gastric cancer in the mammal. In some embodiments, the levels of expression of all the genes in FIG. 1 are detected, wherein substantially increased expression of genes 72-102 in FIG. 1 and/or substantially decreased expression of genes 1-71 in FIG. 1 in the tissue suspected of being cancerous as compared to the adjacent tissue are indicative of presence of intestinal gastric cancer in the mammal.

In another aspect, the invention provides a method for diagnosing intestinal gastric cancer in a mammal, comprising: (a) detecting levels of expression of at least two genes shown in FIG. 2 in an intestinal gastric tissue suspected of being cancerous of the mammal; (b) detecting levels of expression of the genes in an intestinal gastric tissue adjacent to the gastric tissue suspected of being cancerous; (c) comparing the levels of expression of the genes in the gastric tissue suspected of being cancerous to levels of expression of the genes in a normal intestinal gastric mucosa tissue of the same mammal species; (d) comparing the levels of expression of the genes in the adjacent gastric tissue to levels of expression of the genes in a normal intestinal gastric mucosa tissue of the same mammal species; and (e) comparing the levels of expression of the genes in the gastric tissue suspected of being cancerous to the levels of expression of the genes in the adjacent gastric tissue; wherein substantial variance of the levels of expression of the genes shown in FIG. 2 between the gastric tissue suspected of being cancerous and the normal gastric mucosa tissue and between the adjacent gastric tissue and the normal gastric mucosa tissue, and no substantial variance of the levels of expression of the genes shown in FIG. 2 between the gastric tissue suspected of being cancerous and the adjacent gastric tissue are indicative of presence of intestinal gastric cancer in the mammal.

In some embodiments, the levels of expression of at least 5, at least 10, at least 20, at least 30, at least 40, at least 50, at least 60, at least 70, or at least 80 genes in FIG. 2 are detected. In some embodiments, the levels of expression of all the genes in FIG. 2 are detected, wherein substantially decreased expression of at least one of genes 1-53 in FIG. 2 and substantially increased expression of at least one of genes 54-84 in FIG. 2 in the gastric tissue suspected of being cancerous and the adjacent tissue as compared to the normal gastric mucosa tissue are indicative of presence of intestinal gastric cancer in the mammal. In some embodiments, the levels of expression of all the genes in FIG. 2 are detected, wherein substantially decreased expression of at least 2, at least 5, at least 10, at least 20, at least 30, at least 31, at least 40, or at least 50 of genes 1-53 in FIG. 2 and/or substantially increased expression of at least 2, at least 5, at least 10, at least 18, at least 20, at least 30 of genes 54-84 in FIG. 2 in the gastric tissue suspected of being cancerous and the adjacent tissue as compared to the normal gastric mucosa tissue are indicative of presence of intestinal gastric cancer in the mammal. In some embodiments, the levels of expressions of all the genes in FIG. 2 are detected, wherein substantially decreased expression of genes 1-53 in FIG. 2 and/or substantially increased expression of genes 54-84 in FIG. 2 in the gastric tissue suspected of being cancerous and the adjacent tissue as compared to the normal gastric mucosa tissue are indicative of presence of intestinal gastric cancer in the mammal.

In another aspect, the invention provides a method for assessing levels of differentiation of intestinal gastric cancer in a mammal, comprising: (a) detecting levels of expression of at least two genes shown in FIG. 3 in an intestinal gastric cancer tissue of the mammal; (b) detecting levels of expression of the genes in an intestinal gastric tissue adjacent to the cancer tissue; and (c) comparing the levels of expression of the genes in the cancer tissue to the levels of expression of the genes in the adjacent tissue; wherein substantial variance of the levels of expression of the genes between the cancer tissue and the adjacent tissue is indicative of high level of differentiation of the intestinal gastric cancer in the mammal.

In some embodiments, the levels of expression of at least 5, at least 10, at least 20, at least 30, at least 40, or at least 50 genes in FIG. 3 are detected. In some embodiments, the levels of expression of all genes shown in FIG. 3 are detected, wherein substantially increased expression of at least one of genes 1-16 shown in FIG. 3 and substantially decreased expression of at least one of genes 17-55 shown in FIG. 3 in the cancer tissue as compared to the adjacent tissue are indicative of high level of differentiation in the intestinal gastric cancer tissue. In some embodiments, the levels of expression of all genes shown in FIG. 3 are detected, wherein substantially increased expression of at least 2, at least 3, at least 5, at least 9, at least 10, or at least 15 of genes 1-16 in FIG. 3 and/or substantially decreased expression of at least 2, at least 3, at least 5, at least 10, at least 15, at least 20, at least 23, at least 25, at least 30, or at least 35 of genes 17-55 in FIG. 3 in the cancer tissue as compared to the adjacent tissue are indicative of high level of differentiation in the intestinal gastric cancer tissue. In some embodiments, the levels of expression of all genes in FIG. 3 are detected, wherein substantially increased expression of genes 1-16 in FIG. 3 and/or substantially decreased expression of genes 17-55 in FIG. 3 in the cancer tissue as compared to the adjacent tissue are indicative of high level of differentiation in the intestinal gastric cancer tissue.

In another aspect, the invention provides a method for assessing levels of differentiation of intestinal gastric cancer in a mammal, comprising: (a) detecting levels of expression of at least two genes shown in FIG. 4 in an intestinal gastric cancer tissue of the mammal; (b) detecting levels of expression of the genes in an intestinal gastric tissue adjacent to the cancer tissue; and (c) comparing the levels of expression of the genes in the cancer tissue to the levels of expression of the genes in the adjacent tissue; wherein substantial variance of the levels of expression of the genes between the cancer tissue and the adjacent tissue is indicative of low level of differentiation of the intestinal gastric cancer in the mammal.

In some embodiments, the levels of expression of at least 5, at least 10, at least 20, at least 30, or at least 40 genes in FIG. 4 are detected. In some embodiments, the levels of expression of all genes shown in FIG. 4 are detected, wherein substantially increased expression of at least one of genes 1-28 in FIG. 4 and substantially decreased expression of at least one of genes 29-46 in FIG. 4 in the cancer tissue as compared to the adjacent tissue are indicative of low level of differentiation in the intestinal gastric cancer tissue. In some embodiments, the levels of expression of all genes shown in FIG. 4 are detected, wherein substantially increased expression of at least 2, at least 3, at least 5, at least 10, at least 15, at least 16, at least 20, or at least 25 of genes 1-28 in FIG. 4 and/or substantially decreased expression of at least 2, at least 3, at least 5, at least 10, or at least 15 of genes 29-46 in FIG. 4 in the cancer tissue as compared to the adjacent tissue are indicative of low level of differentiation in the intestinal gastric cancer tissue. In some embodiments, the levels of expression of all genes shown in FIG. 4 are detected, wherein substantially increased expression of genes 1-29 in FIG. 4 and/or substantially decreased expression of genes 30-48 in FIG. 4 in the cancer tissue as compared to the adjacent tissue are indicative of low level of differentiation in the intestinal gastric cancer tissue.

In another aspect, the invention provides systems for diagnosing intestinal gastric cancer and/or assessing levels of differentiation of intestinal gastric cancer. In some embodiments, the invention provides a system for diagnosing intestinal gastric cancer, consisting essentially of at least two isolated polynucleotide molecules and wherein each isolated polynucleotide molecule is capable of detecting expression of a different gene, wherein each gene is selected from the group consisting of genes 1-102 shown in FIG. 1. In some embodiments, the system consists essentially of at least 5, at least 10, at least 20, at least 30, at least 40, at least 50, at least 60, at least 61, at least 70, at least 80, at least 90, at least 100, at least 102 of isolated polynucleotide molecules, wherein each isolated polynucleotide molecule is capable of detecting expression of a different gene and wherein each gene is selected from the group consisting of genes 1-102 shown in FIG. 1. In some embodiments, the system consists essentially of a plurality of isolated polynucleotide molecules, wherein each isolated polynucleotide molecule is capable of detecting expression of a gene selected from the group consisting of at least 61, at least 70, at least 80, at least 90, at least 100, or 102 genes shown in FIG. 1, whereby differential expression of said genes can be detected.

In some embodiments, the invention provides a system for diagnosing intestinal gastric cancer, consisting essentially of at least two isolated polynucleotide molecules, wherein each isolated polynucleotide molecule is capable of detecting expression of a different gene, wherein each gene is selected from the group consisting of genes 1-84 shown in FIG. 2. In some embodiments, the system consists essentially of at least 5, at least 10, at least 20, at least 30, at least 40, at least 50, at least 60, at least 70, at least 80, at least 84 of isolated polynucleotide molecules, wherein each isolated polynucleotide molecule is capable of detecting expression of a different gene and wherein each gene is selected from the group consisting of genes 1-84 shown in FIG. 2. In some embodiments, the system consists essentially of a plurality of isolated polynucleotide molecules, wherein each isolated polynucleotide molecule is capable of detecting expression of a gene selected from the group consisting of at least 50, at least 60, at least 70, or 84 genes shown in FIG. 2, whereby differential expression of said genes can be detected.

In another aspect, the invention provides a system for assessing levels of differentiation of intestinal gastric cancer in a mammal, consisting essentially of at least two isolated polynucleotide molecules, wherein each isolated polynucleotide molecule is capable of detecting expression of a different gene, wherein each gene is selected from the group consisting of genes 1-55 shown in FIG. 3. In some embodiments, the system consists essentially of at least 5, at least 10, at least 20, at least 30, at least 33, at least 40, at least 50, at least 55 of isolated polynucleotide molecules wherein each isolated polynucleotide molecule is capable of detecting expression of a different gene and wherein each gene is selected from the group consisting of genes 1-55 shown in FIG. 3. In some embodiments, the system consists essentially of a plurality of isolated polynucleotide molecules, wherein each isolated polynucleotide molecule is capable of detecting expression of a gene selected from the group consisting of at least 33, at least 40, at least 50, or 55 genes shown in FIG. 3, whereby differential expression of said genes can be detected.

In another aspect, the invention provides a system for assessing levels of differentiation of intestinal gastric cancer in a mammal, consisting essentially of at least two isolated polynucleotide molecules, wherein each isolated polynucleotide molecule is capable of detecting expression of a different gene, wherein each gene is selected from the group consisting of genes 1-46 shown in FIG. 4. In some embodiments, the system consists essentially of at least 5, at least 10, at least 20, at least 27, at least 30, at least 40, or at least 46 of isolated polynucleotide molecules, wherein each isolated polynucleotide molecule is capable of detecting expression of a gene and wherein each gene is selected from the group consisting of genes 1-46 shown in FIG. 4. In some embodiments, the system consists essentially of a plurality of isolated polynucleotide molecules, wherein each isolated polynucleotide molecule is capable of detecting expression of a gene selected from the group consisting of at least 27, at least 30, at least 40, or 46 genes shown in FIG. 4, whereby differential expression of said genes can be detected.

In another aspect, the invention provides a kit for diagnosing intestinal gastric cancer and/or assessing levels of differentiation of intestinal gastric cancer. The kits comprise, for example, one or more systems described herein.

The levels of gene expression for methods described herein may be detected using any methods. In some embodiments, the levels of gene expression are detected by detecting the levels of mRNA encoded by the gene. In some embodiments, the levels of gene expression are detected by detecting the levels of the protein encoded by the gene.

The isolated polynucleotide molecules in the systems described herein may be DNA (e.g., synthetic, genomic, cDNA), RNA, or PNA. In some embodiments, the isolated polynucleotide molecules are immobilized on an array. The array may be a chip array, a plate array, a bead array, a pin array, or a membrane array. The array may also be a solid surface array or a liquid array. The array may also be an oligonucleotide array, a polynucleotide array, a cDNA array.

The methods, systems, and kits described herein may also be used for monitoring the progression of intestinal gastric cancer.

### DESCRIPTION OF FIGURES

FIG. 1 provides a list of genes that are differentially expressed in intestinal gastric cancer tissues as compared to corresponding adjacent intestinal gastric tissues.

FIG. 2 provides a list of genes that are differentially expressed in intestinal gastric cancer tissues and the corresponding adjacent intestinal gastric tissues as compared to normal gastric mucosa tissues.

FIG. 3 provides a list of genes that are differentially expressed in highly differentiated intestinal gastric cancer tissues as compared to corresponding adjacent intestinal gastric tissues.

FIG. 4 provides a list of genes that are differentially expressed in poorly differentiated intestinal gastric cancer tissues as compared to corresponding adjacent intestinal gastric tissues.

FIG. 5 provides information about 21 patients from whom intestinal gastric cancer tissues and corresponding adjacent intestinal gastric tissues were obtained.

FIG. 6 provides a schematic diagram of a cDNA labeling process.

FIG. 7 provides additional differential expression data of the THY gene (GB accession: AK057865) based on RT/PCR and tissue array.

### DETAILED DESCRIPTION OF THE INVENTION

The invention is based on gene expression studies of intestinal gastric cancer tissue samples and their corresponding adjacent intestinal gastric tissue samples from cancer patients, and normal human gastric mucosa tissue samples. Specifically, using DNA oligonucleotide microarrays, we have compared the gene expression profiles of the different tissue samples. We have identified 102 genes that were either overexpressed or underexpressed in intestinal gastric cancer tissues as compared to corresponding adjacent intestinal gastric tissues; 84 genes that were either overexpressed or underexpressed in intestinal gastric cancer tissues and/or corresponding adjacent intestinal gastric cancer tissues as compared to gastric mucosa tissues from normal people; 55 genes that were either overexpressed or underexpressed in highly differentiated cancer tissues as compared to corresponding adjacent intestinal gastric tissues; and 46 genes that were either overexpressed or underexpressed in poorly differentiated cancer tissues as compared to corresponding adjacent intestinal gastric tissues.

Accordingly, the invention provides methods of detecting differential gene expression in an intestinal gastric tissue in an individual, methods of diagnosing intestinal gastric cancer, and methods of assessing levels of differentiation of intestinal gastric cancer tissues, by detecting expression levels of two or more of the genes identified herein. The invention also provides systems for detecting gene expression consisting essentially of a plurality (i.e., at least two) of isolated molecules, each molecule is capable of detecting a gene identified herein. The invention further provides kits useful for methods described herein.

### General Techniques

The practice of the present invention will employ, unless otherwise indicated, conventional techniques of molecular biology (including recombinant techniques), microbiology, cell biology, and biochemistry, which are within the skill of the art. Such techniques are explained fully in the literature, such as: Molecular Cloning: A Laboratory Manual, vol. 1-4, third edition (Sambrook et al., 2001); Oligonucleotide Synthesis (M. J. Gait, ed., 1984); Methods in Enzymology (Academic Press, Inc.); Current Protocols in Molecular Biology (F. M. Ausubel et al., eds., 1987); PCR Cloning Protocols, (Yuan and Janes, eds., 2002, Humana Press).

In addition to the above references, protocols for in vitro amplification techniques, such as the polymerase chain reaction (PCR), the ligase chain reaction (LCR), Qβ-replicase amplification, and other RNA polymerase mediated techniques (e.g., NASBA), useful, e.g., for amplifying oligonucleotide probes of the invention, are found in Mullis et al., (1987) U.S. Pat. No. 4,683,202; PCR Protocols: A Guide to Methods and Applications (Innis et al., eds.) Academic Press, Inc., San Diego, Calif. (1990); Anheim and Levinson (1990) C&EN 36; The Journal of NIH Research (1991) 3:81; Kwoh et al. (1989) Proc Natl Acad Sci USA 86:1173; Guatelli et al. (1990) Proc Natl Acad Sci USA 87:1874; Lomell et al. (1989) J Clin Chem 35:1826; Landegren et al. (1988) Science 241:1077; Van Brunt (1990) Biotechnology 8:291; Wu and Wallace (1989) Gene 4:560; Barringer et al. (1990) Gene 89:117; Sooknanan and Malek (1995) Biotechnology 13:563. Additional methods, useful for cloning nucleic acids, include Wallace et al., U.S. Pat. No. 5,426,039. Improved methods of amplifying large nucleic acids by PCR are summarized in Cheng et al. (1994) Nature 369:684, and the references therein.

### DEFINITIONS

Unless defined otherwise, all scientific and technical terms are understood to have the same meaning as commonly used in the art to which they pertain. For the purpose of the present invention, the following terms are defined below.

As used herein, “individual” refers to a mammal, such as a human, a nonhuman primate, an experimental animal, such as a mouse or rat, a pet animal, such as a cat or dog, or a farm animal, such as a horse, sheep, cow, or pig.

“Intestinal gastric tissue adjacent to the tissue suspected of being cancerous,” “corresponding adjacent intestinal gastric tissue,” or “adjacent tissue” as used herein refers to the intestinal gastric tissue of an individual that is about 5 cm or more away from any part of the tissue that is suspected of being cancerous (including a tissue that is cancerous). The adjacent tissue is typically normal based on morphological or pathological criteria.

“A normal intestinal gastric mucosa tissue” refers to an intestinal gastric mucosa tissue obtained from an individual who is healthy with respect to a specific disease factor or criterion. It will be appreciated that the term “normal” as used herein is relative to a specified disease condition, or criterion. Thus, an individual described as healthy with reference to any specified disease or disease criterion can be diagnosed with any other one or more disease, or may exhibit any other one or more disease criterion.

The term “healthy individual,” or “normal individual” as used herein, is relative to a specified disease or disease criterion, e.g., the individual does not exhibit the specified disease criterion or is not diagnosed with the specified disease. It will be understood that the individual in question can exhibit symptoms, or possess various indicator factors, for another disease.

As used herein, “differential expression” refers to increased or decreased production of a gene expression product, i.e., the gene is either overexpressed or underexpressed. Differential expression may be assessed qualitatively (i.e., by determining the presence or absence of a gene product) and/or quantitatively (i.e., by determining the increase or decrease of a gene product in a relative amount).

When referring to a pattern of expression, a “qualitative” difference in gene expression refers to a difference that is not assigned a relative value. That is, such a difference is designated by an “all or nothing” valuation. Such an all or nothing variation can be, for example, expression above or below a threshold of detection (an on/off pattern of expression). Alternatively, a qualitative difference can refer to expression of different types of expression products, e.g., different alleles (e.g., a mutant or polymorphic allele), variants (including sequence variants as well as post-translationally modified variants), etc.

In contrast, a “quantitative” difference, when referring to a pattern of gene expression, refers to a difference in expression that can be assigned a numerical value, such as a value on a graduated scale, (e.g., a 0-5 or 1-10 scale, a +−+++ scale, a grade 1-grade 5 scale, or the like; it will be understood that the numbers selected for illustration are entirely arbitrary and in no-way are meant to be interpreted to limit the invention).

“Substantial variance” in levels of expression refers to levels of expression that are at least about two times different. For example, a “substantially increased expression” refers to an expression levels that is at least about twice as high as the expression levels being compared to. A “substantially decreased expression” refers to an expression level that is at most about half as low as the expression levels being compared to.

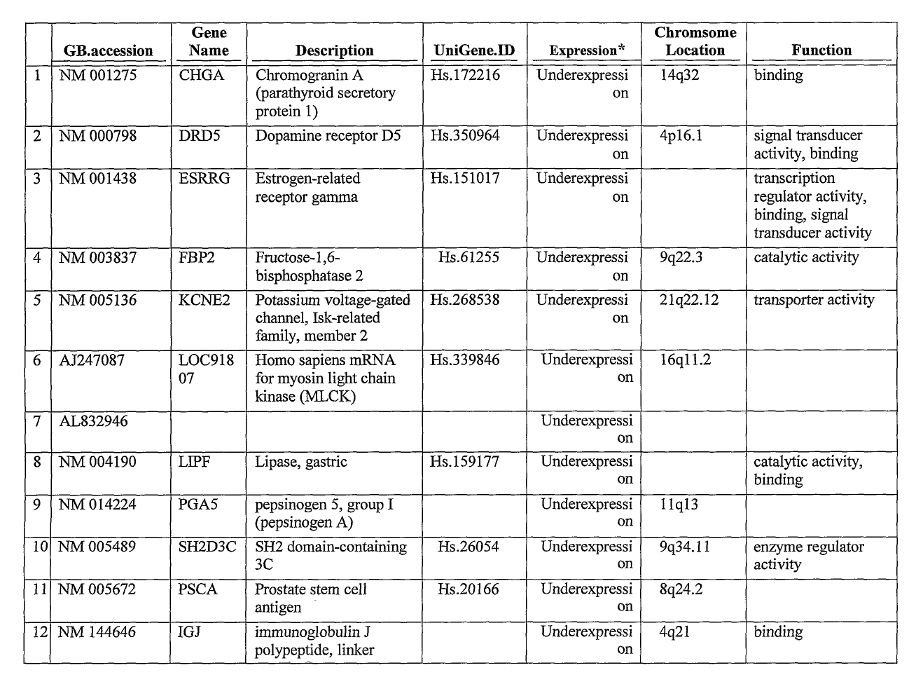

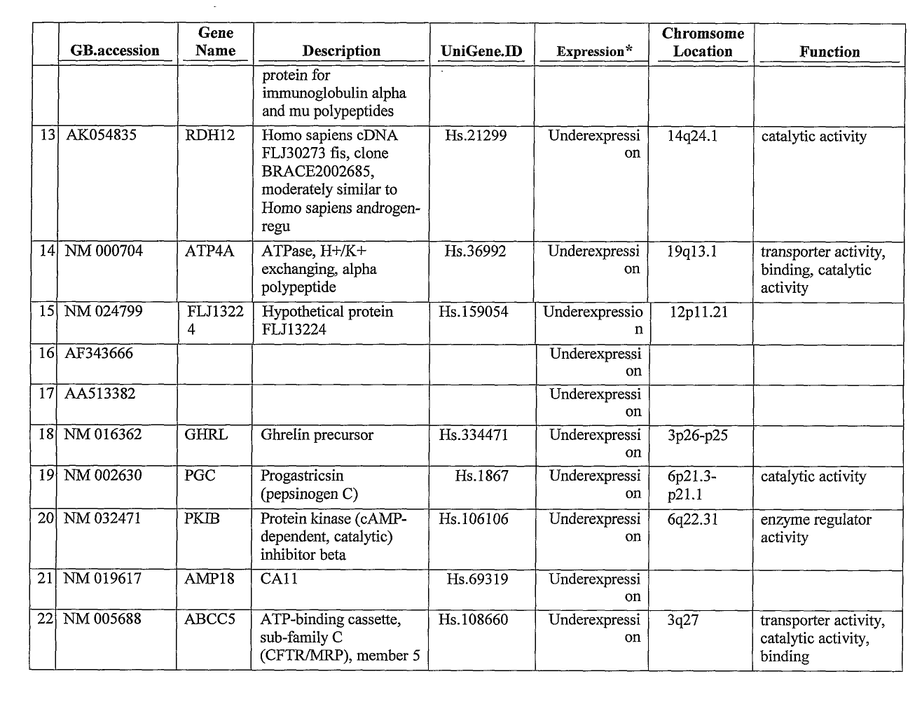

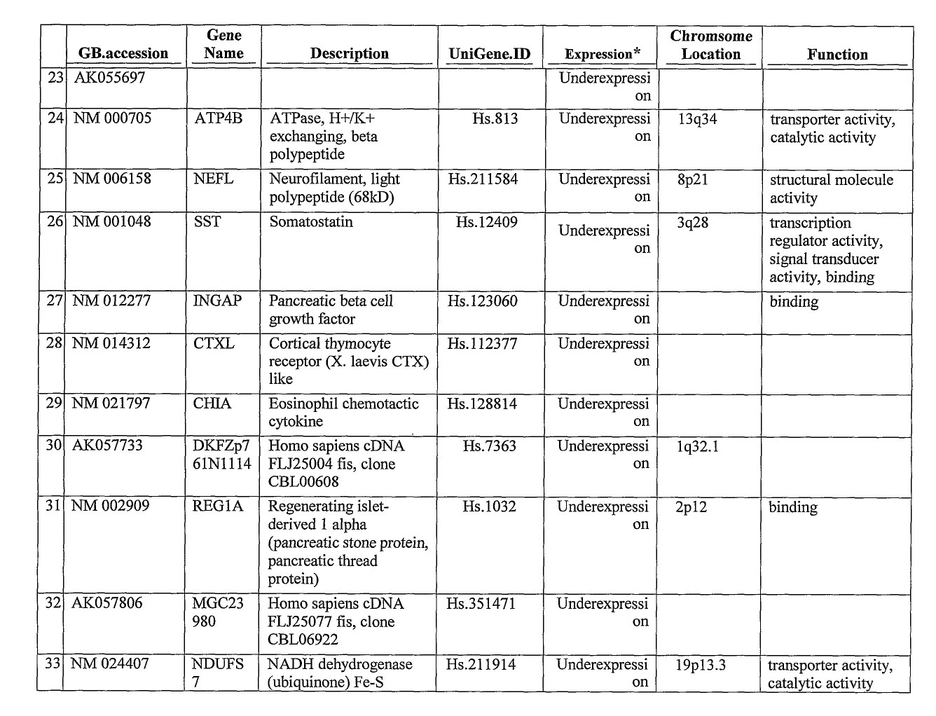

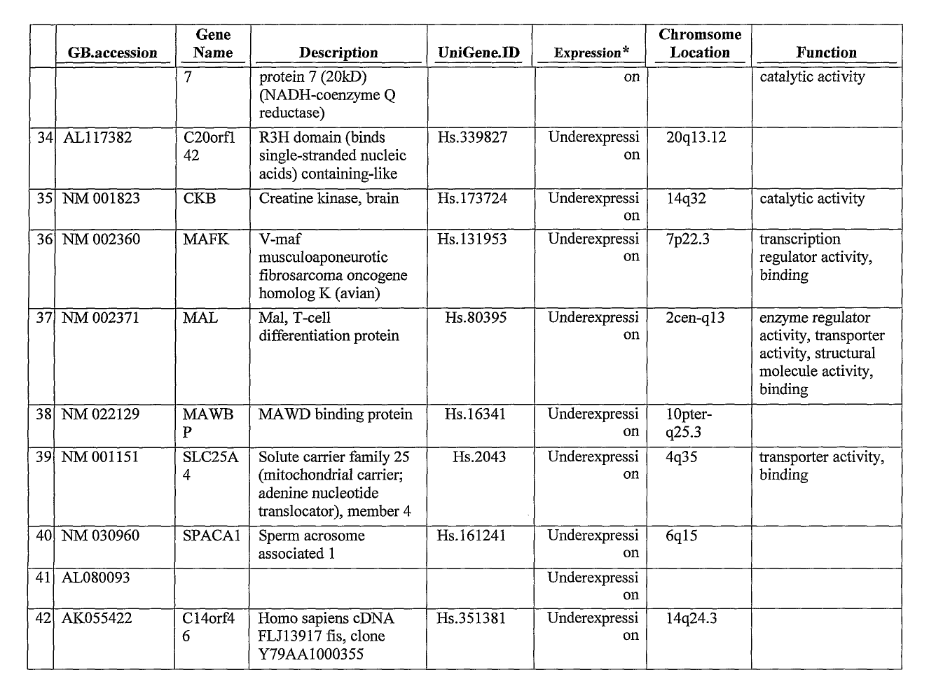

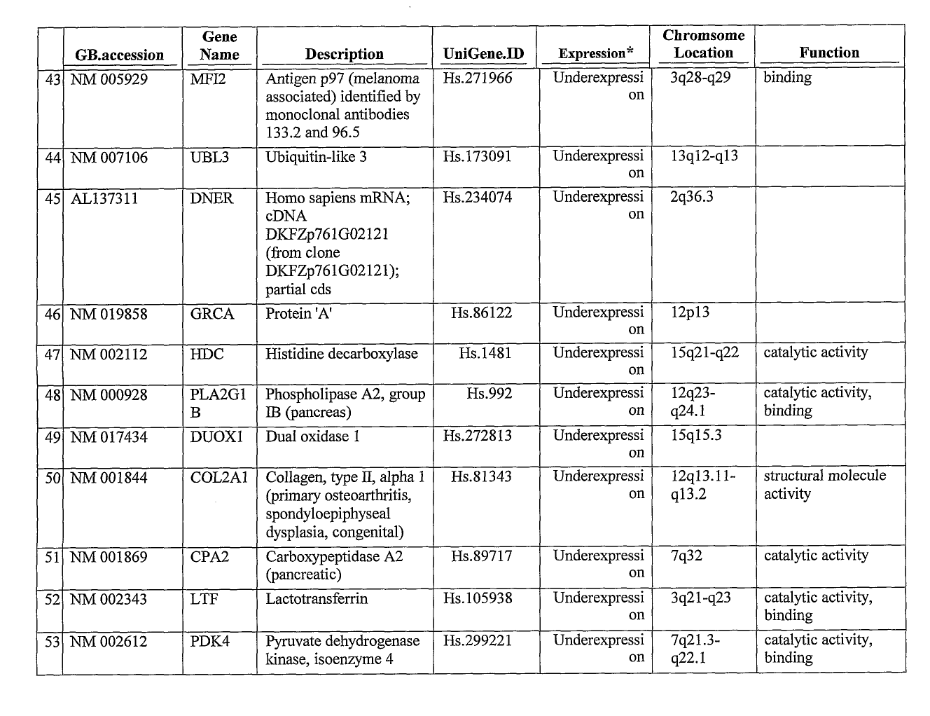

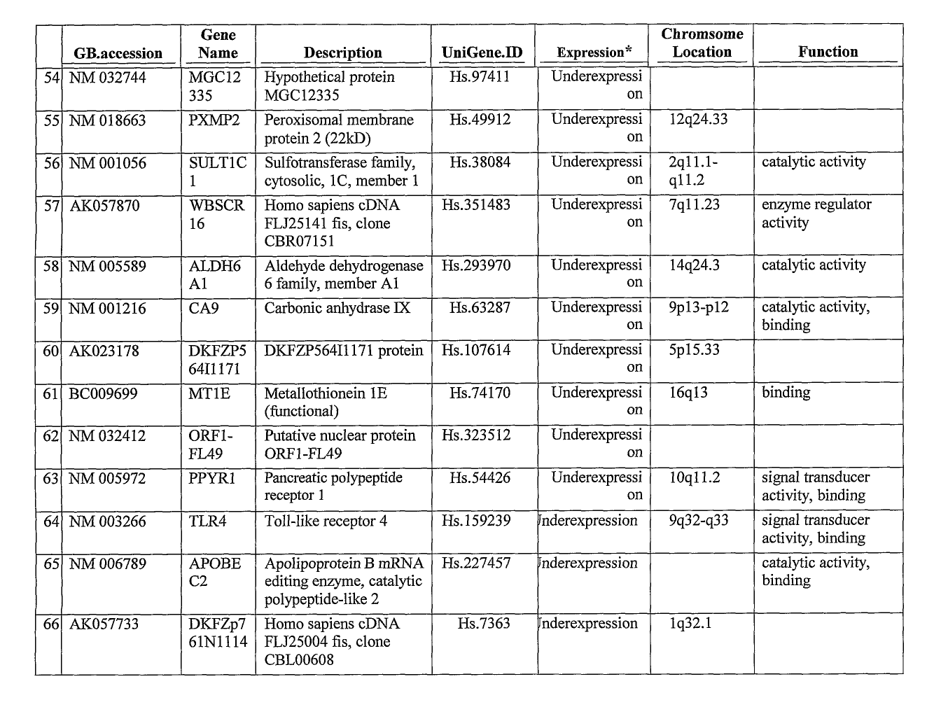

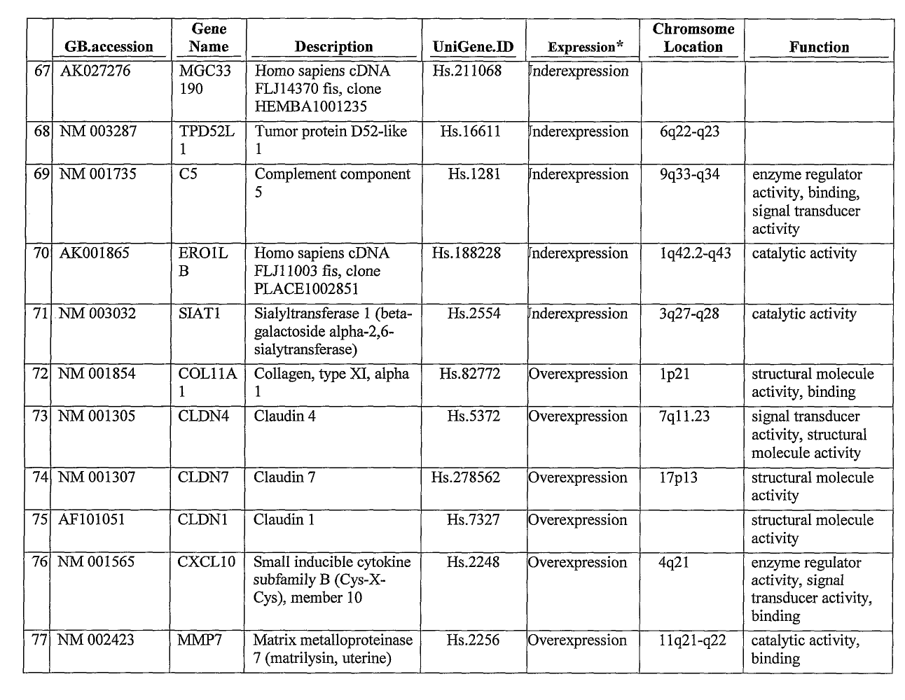

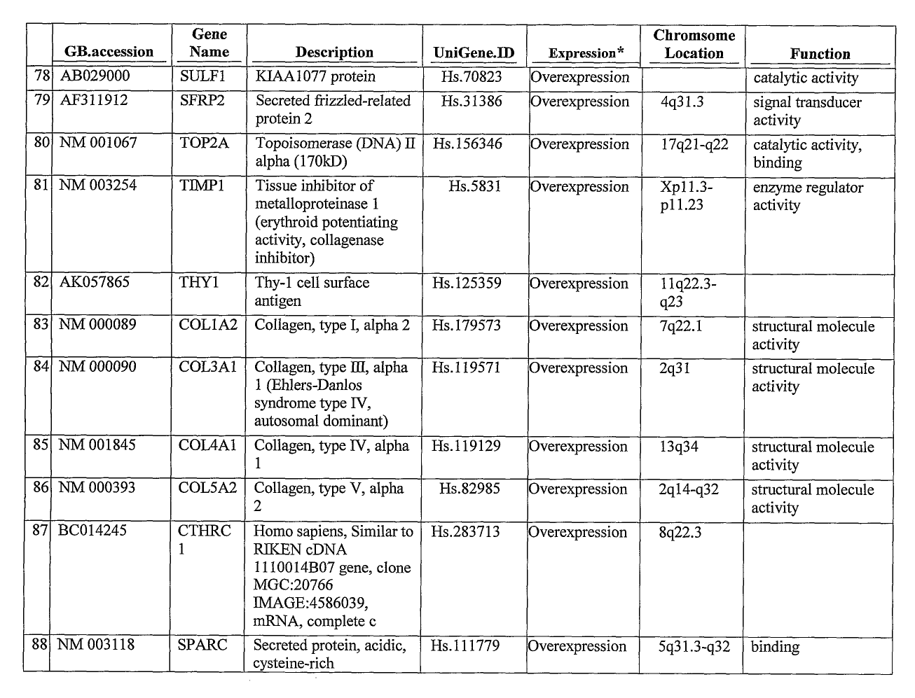

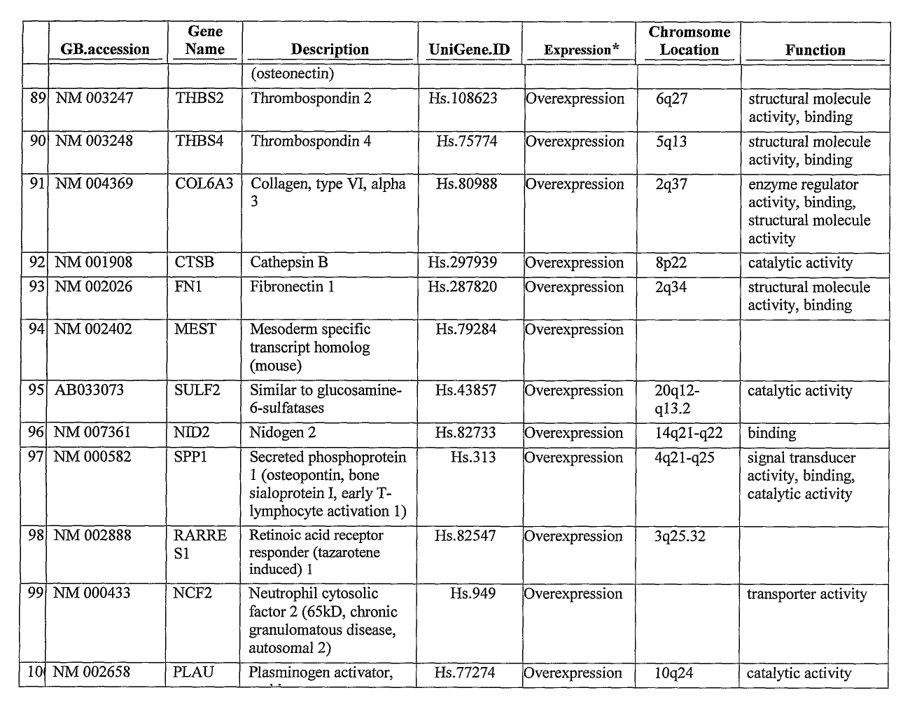

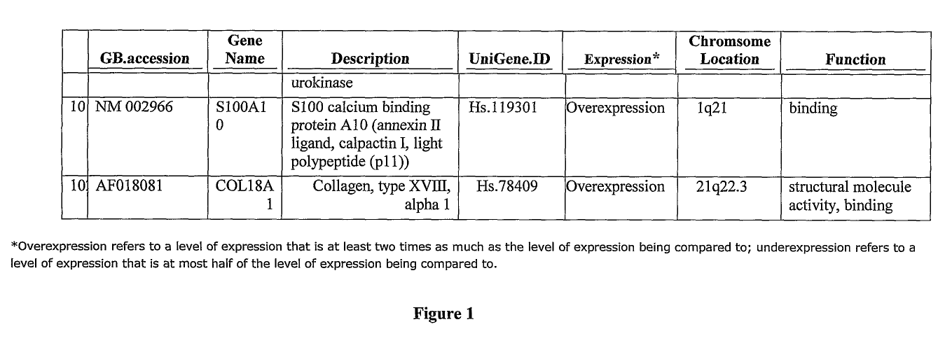

The term “expression profile” refers to the collection of expression values for a plurality (e.g., at least 2, at least 5, at least about 10, about 30, about 100, about 200, or more) of genes listed in FIGS. 1-4. In many cases, the expression profile represents the expression pattern for all of the genes. Alternatively, the expression profile represents the expression pattern for one or more subsets of the genes. As used herein, the term “gene expression system” or “system for detecting gene expression” refers to any system, device or means to detect gene expression.

The term “diagnostic oligonucleotide set” or “oligonucleotide set” generally refers to a set of two or more oligonucleotides that, when evaluated for differential expression of their products, collectively yields predictive data. Such predictive data typically relates to diagnosis, prognosis, monitoring of therapeutic outcomes, and the like. In general, the components of a diagnostic oligonucleotide set are distinguished from nucleotide sequences that are evaluated by analysis of the DNA to directly determine the genotype of an individual as it correlates with a specified trait or phenotype, such as a disease, in that it is the pattern of expression of the components of the diagnostic nucleotide set, rather than mutation or polymorphism of the DNA sequence that provides predictive value. It will be understood that a particular component (or member) of a diagnostic nucleotide set can, in some cases, also present one or more mutations, or polymorphisms that are amenable to direct genotyping by any of a variety of well known analysis methods, e.g., Southern blotting, RFLP, AFLP, SSCP, SNP, and the like.

The terms “oligonucleotide” and “polynucleotide” and “nucleic acid,” used interchangeably herein, refer to a polymeric form of two or more nucleotides of any length and any three-dimensional structure (e.g., single-stranded, double-stranded, triple-helical, etc.), which contain deoxyribonucleotides, ribonucleotides, and/or analogs or modified forms of deoxyribonucleotides or ribonucleotides. Nucleotides may be DNA or RNA, and may be naturally occurring, or synthetic, or non-naturally occurring. A nucleic acid of the present invention may contain phosphodiester bonds or an alternate backbone, comprising, for example, phosphoramide, phosphorothioate, phosphorodithioate, O-methylphosphoroamidite linkages, and peptide nucleic acid backbones and linkages. The term polynucleotide includes peptide nucleic acids (PNA).

The terms “polypeptide,” “peptide,” and “protein” are used interchangeably herein to refer to a polymer of amino acid residues. The terms apply to amino acid polymers in which one or more amino acid residue is an analogue of a corresponding naturally occurring amino acid, as well as to naturally occurring amino acid polymers. The term also includes variants on the traditional peptide linkage joining the amino acids making up the polypeptide.

An “antibody” (interchangeably used in plural form) is an immunoglobulin molecule capable of specific binding to a target, such as a carbohydrate, polynucleotide or polypeptide, through at least one antigen recognition site, located in the variable region of the immunoglobulin molecule. As used herein, the term encompasses not only intact antibodies, but also fragments thereof (such as Fab, Fab′, F(ab′)₂, Fv), single chain (ScFv), mutants thereof, fusion proteins comprising an antibody portion, humanized antibodies, and any other modified configuration of the immunoglobulin molecule that comprises an antigen recognition site of the required specificity.

An “isolated” or “purified” molecule is one that is substantially free of the materials with which it is associated in nature. By substantially free is meant at least 50%, preferably at least 70%, more preferably at least 80%, and even more preferably at least 90% free of the materials with which it is associated in nature.

An “array” is a spatially or logically organized collection, e.g., of oligonucleotide sequences or nucleotide sequence products such as RNA or proteins encoded by an oligonucleotide sequence. In some embodiments, an array includes antibodies or other binding reagents specific for products of a gene.

### Methods of Detecting Differential Gene Expression

The invention provides methods of detecting differential gene expression in intestinal gastric tissue in a mammal comprising (a) detecting levels of expression of at least two of the genes shown in FIG. 1 in an intestinal gastric tissue of the mammal, wherein the tissue is suspected of being cancerous; (b) detecting levels of expression of said genes in an intestinal gastric tissue adjacent to the tissue suspected of being cancerous of the mammal; and c) comparing the levels of expression of said genes between the tissue suspected of being cancerous and the adjacent tissue. In some embodiments, the expression levels of at least 3, at least 4, at least 5, at least 10, at least 20, at least 30, at least 40, at least 50, at least 60, at least 70, at least 80, at least 90, at least 100, or 102 of genes from FIG. 1 are detected.

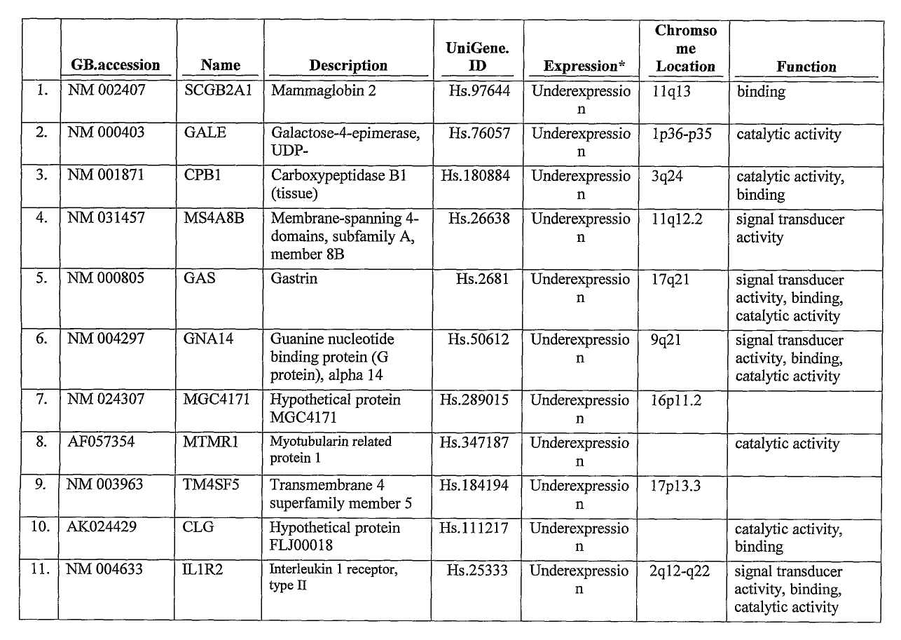

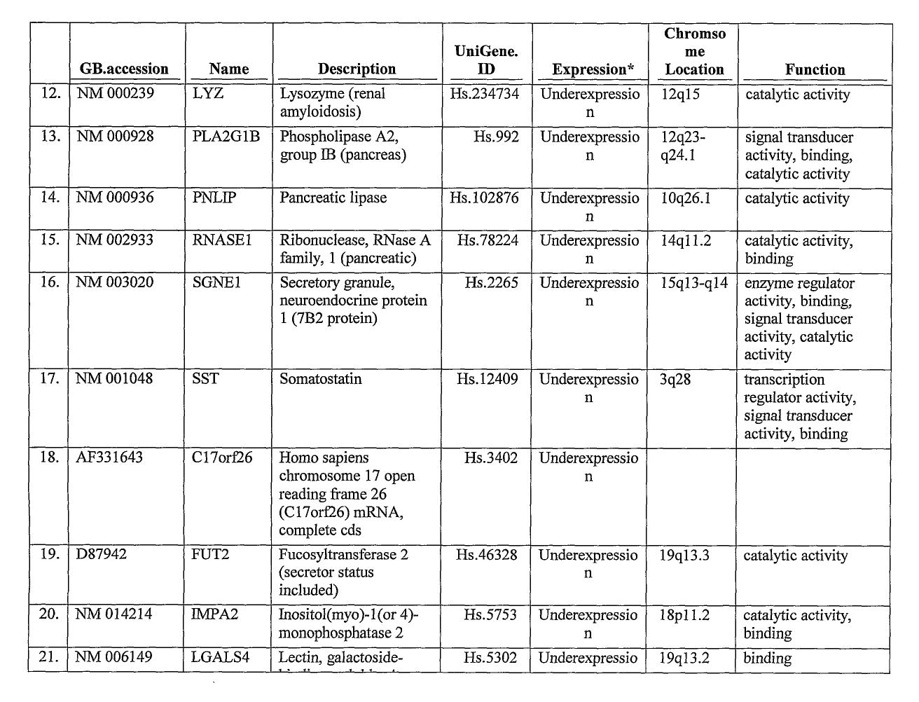

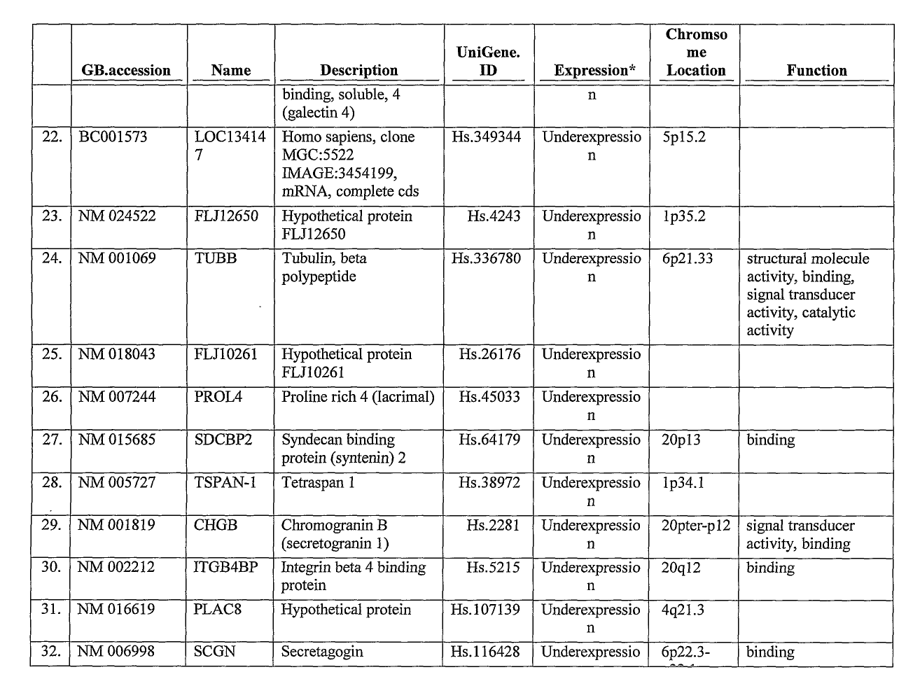

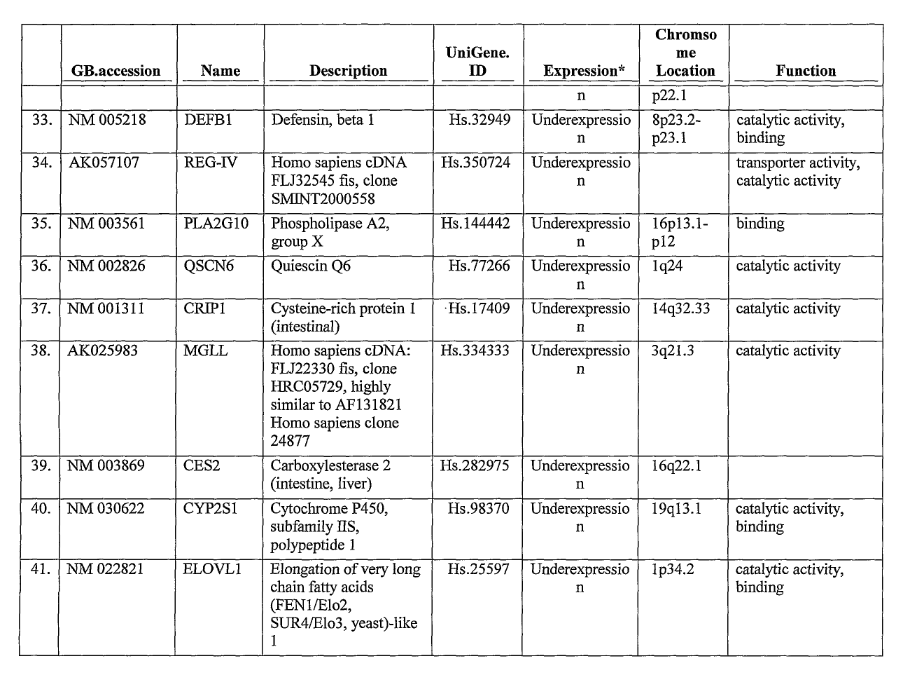

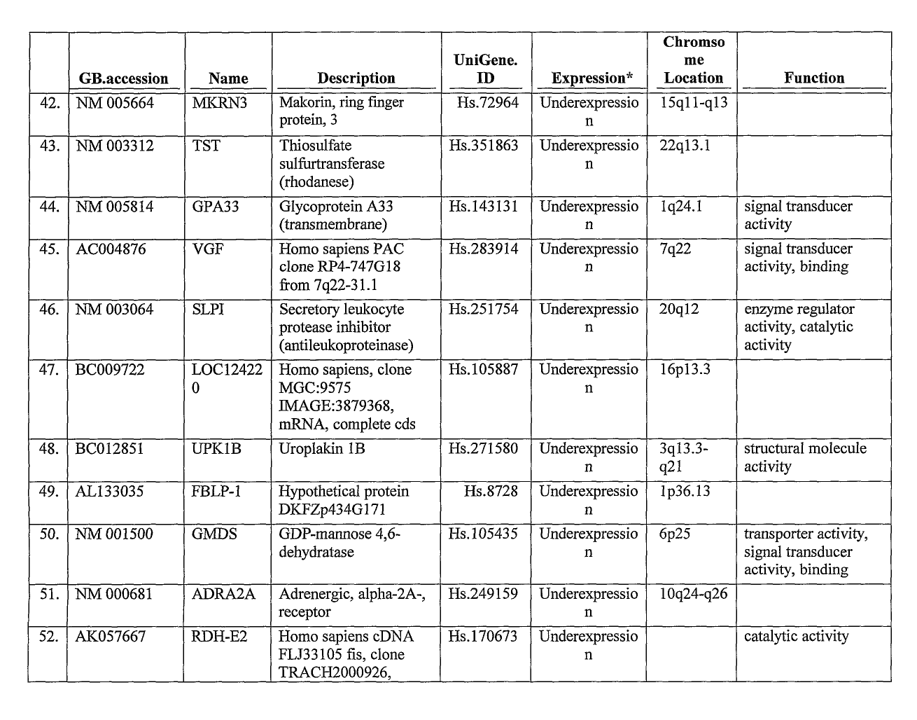

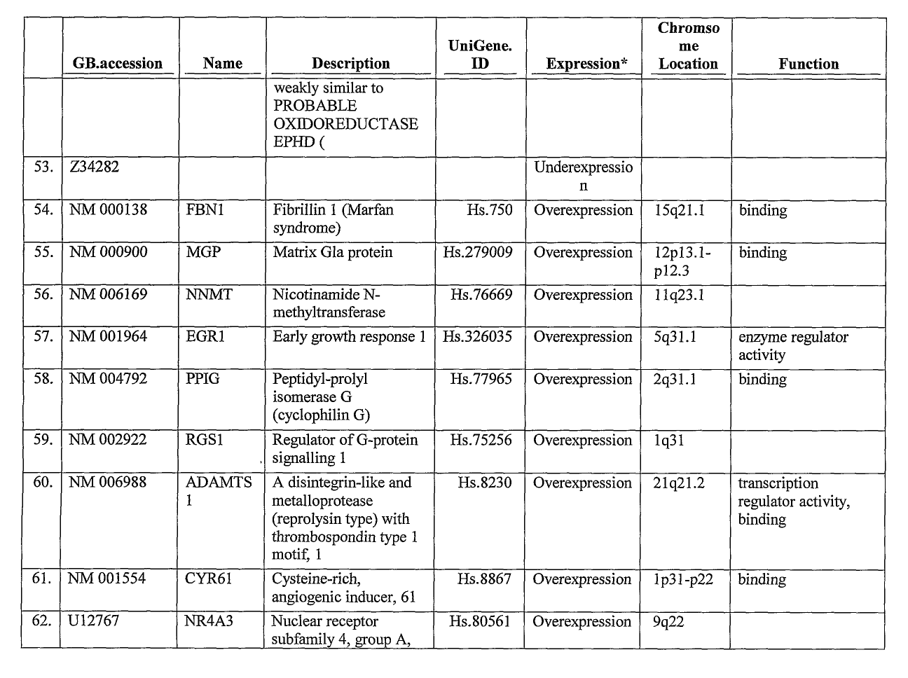

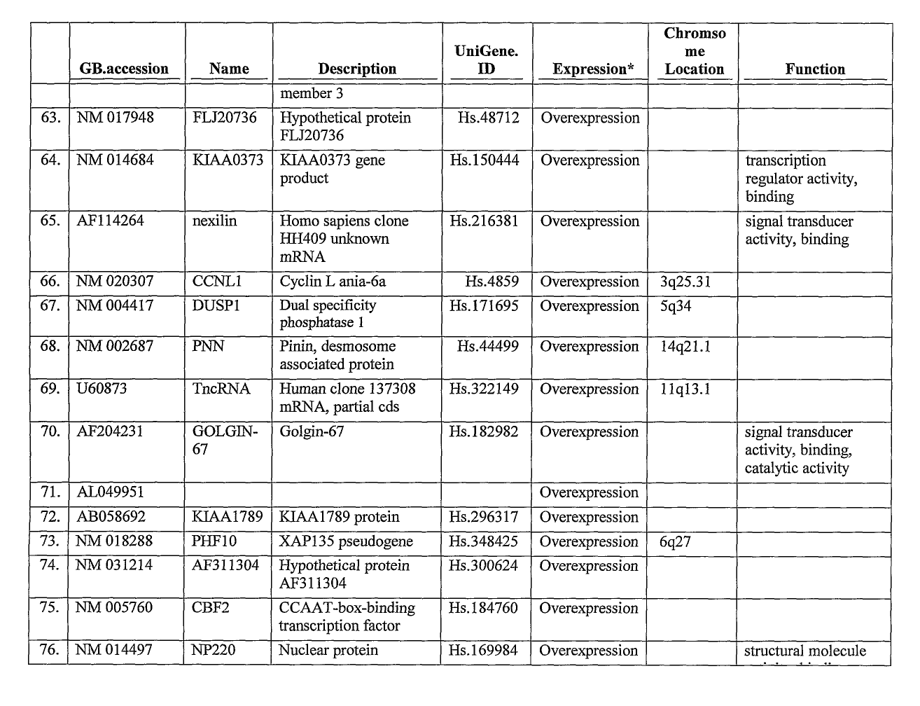

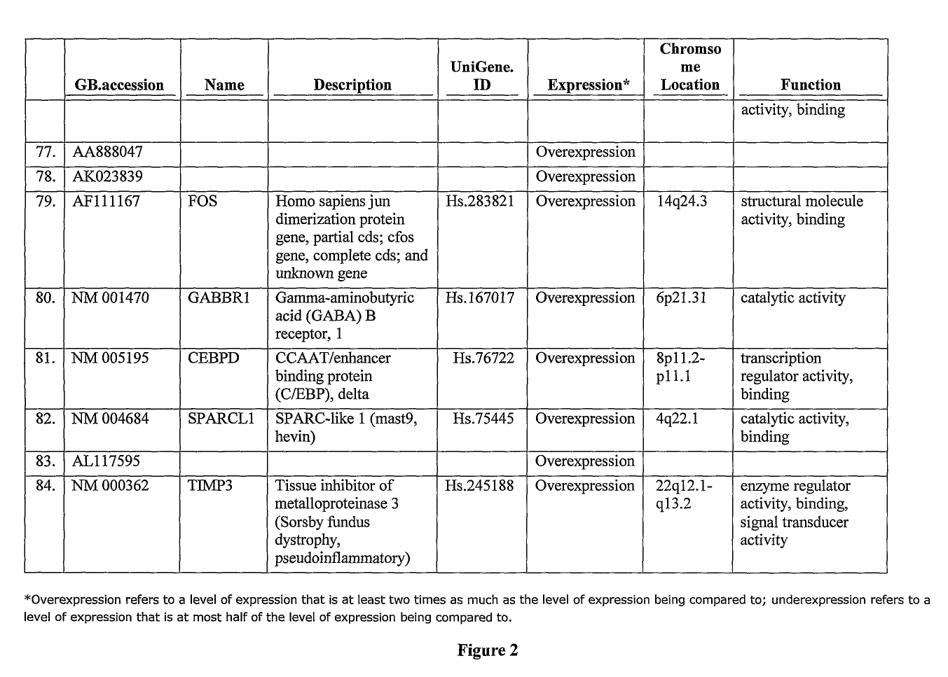

The invention also provides methods of detecting differential gene expression of genes shown in FIG. 2 in a mammal. In some embodiments, the method comprises (a) detecting levels of expression of at least two genes shown in FIG. 2 in an intestinal gastric tissue of the mammal, wherein the tissue is suspected of being cancerous; and (b) comparing the levels of expressions of said genes in the tissue suspected of being cancerous to the levels of expression of the genes in a normal intestinal gastric mucosa tissue of a mammal of the same species. In some embodiments, the method comprises (a) detecting levels of expression of at least two genes shown in FIG. 2 in an intestinal gastric tissue adjacent to an intestinal gastric tissue suspected of being cancerous in a mammal; and (b) comparing the levels of expression of said genes in the adjacent tissue to the levels of expression of genes in a normal intestinal gastric mucosa tissue of a mammal of the same species. In some embodiments, the method comprises (a) detecting levels of expression of at least two of the genes shown in FIG. 2 in an intestinal gastric tissue of the mammal, wherein the tissue is suspected of being cancerous; (b) detecting levels of expression of said genes in an intestinal gastric tissue adjacent to the tissue suspected of being cancerous in the mammal; and c) comparing the levels of expressions of said genes between the tissue suspected of being cancerous (and/or the adjacent tissue) to the levels of expression of genes in a normal intestinal gastric mucosa tissue of a mammal of the same species; and (d) comparing the levels of expression of the genes in the gastric tissue suspected of being cancerous to the levels of expression of genes in the adjacent gastric tissue. In some embodiments, the method comprises (a) detecting levels of expression of at least two genes shown in FIG. 2 in an intestinal gastric tissue of a mammal, wherein the tissue is suspected of being cancerous; (b) detecting levels of expression of said genes in an intestinal gastric tissue adjacent to the gastric tissue suspected of being cancerous; (c) comparing the levels of expression of the genes in the gastric tissue suspected of being cancerous to levels of expression of the genes in a normal intestinal gastric mucosa tissue of the same mammalian species; (d) comparing the levels of expression of the genes in the adjacent gastric tissue to levels of expression of the genes in a normal gastric mucosa tissue of a mammal of the same species; and (e) comparing the levels of expression of the genes in the gastric tissue suspected of being cancerous to the levels of expression of genes in the adjacent gastric tissue.

In some embodiments, the expression levels of at least 3, at least 4, at least 5, at least 10, at least 20, at least 30, at least 40, at least 50, at least 60, at least 70, at least 80, or 84 of genes from FIG. 2 are detected. The expression levels of a plurality of genes (such as at least two of the genes shown in FIG. 2) may constitute an “expression profile” or “molecular signature” that is representative of gene expressions in a sample, and can be used to evaluate the presence, absence, or absolute expression values for a plurality of gene expression products.

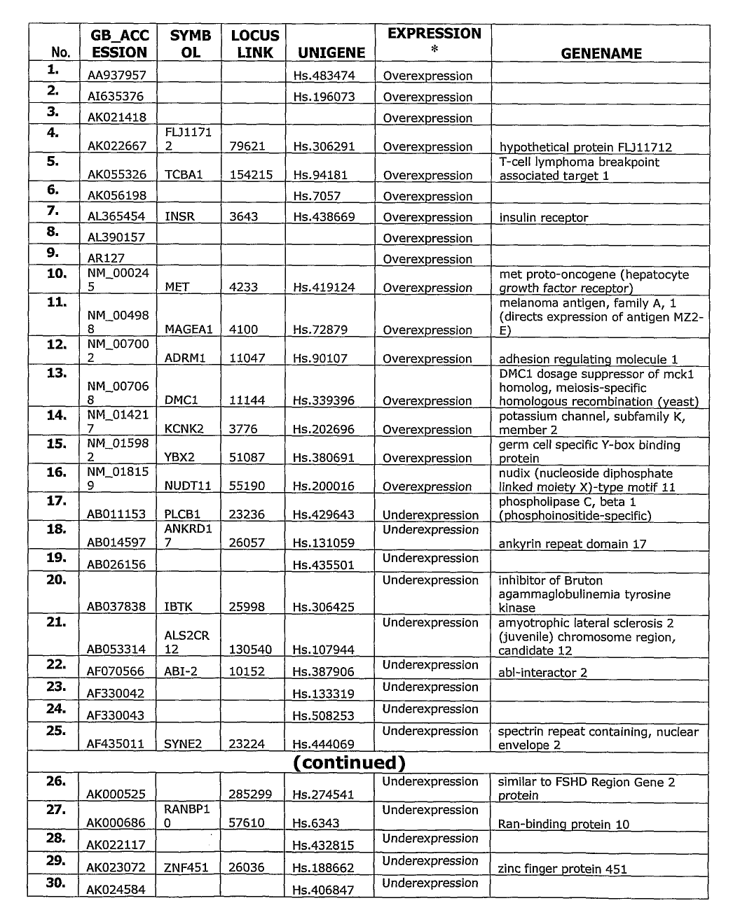

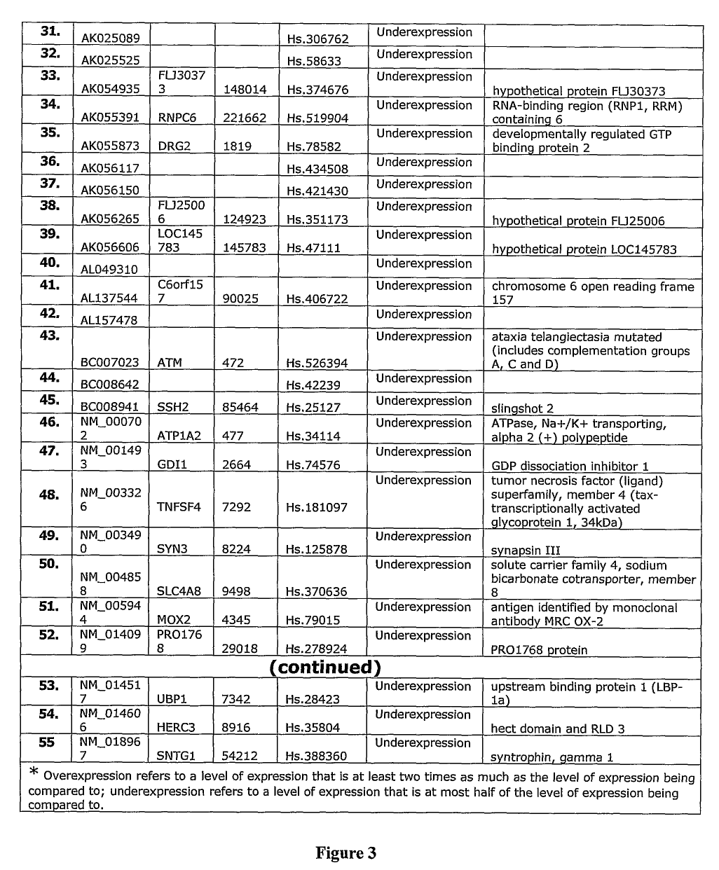

The invention also provides methods of detecting differential gene expression in intestinal gastric tissue in a mammal comprising (a) detecting levels of expression of at least two of the genes shown in FIG. 3 in an intestinal gastric tissue of the mammal, wherein the tissue is suspected of being cancerous; (b) detecting levels of expression of said genes in an intestinal gastric tissue adjacent to the tissue suspected of being cancerous of the mammal; and c) comparing the levels of expressions of said genes between the tissue suspected of being cancerous and the adjacent tissue. In some embodiments, the expression levels of at least 3, at least 4, at least 5, at least 10, at least 20, at least 30, at least 40, at least 50, or 55 of genes from FIG. 3 are detected.

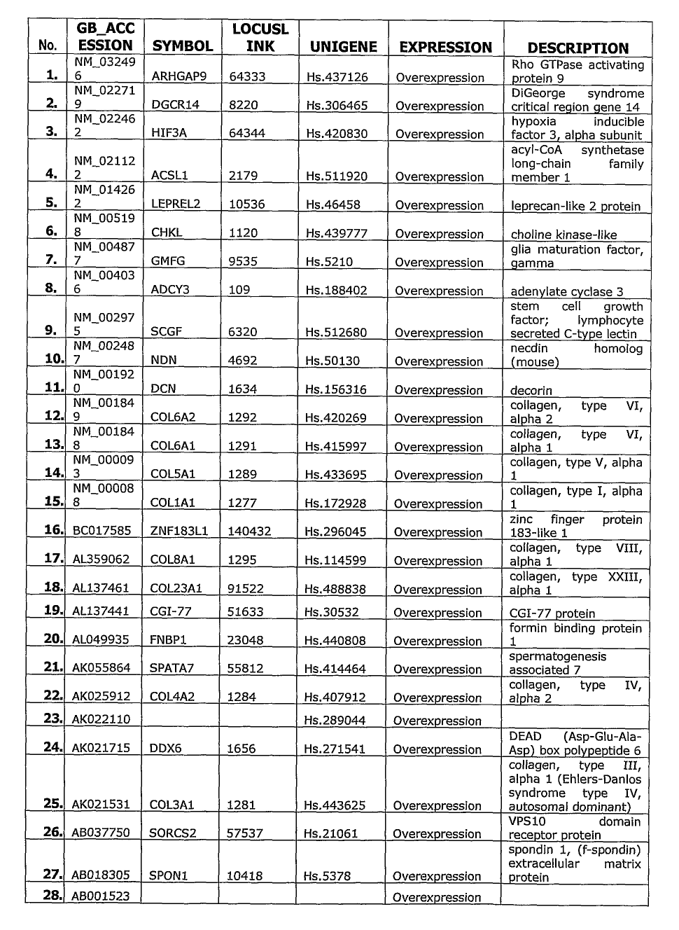

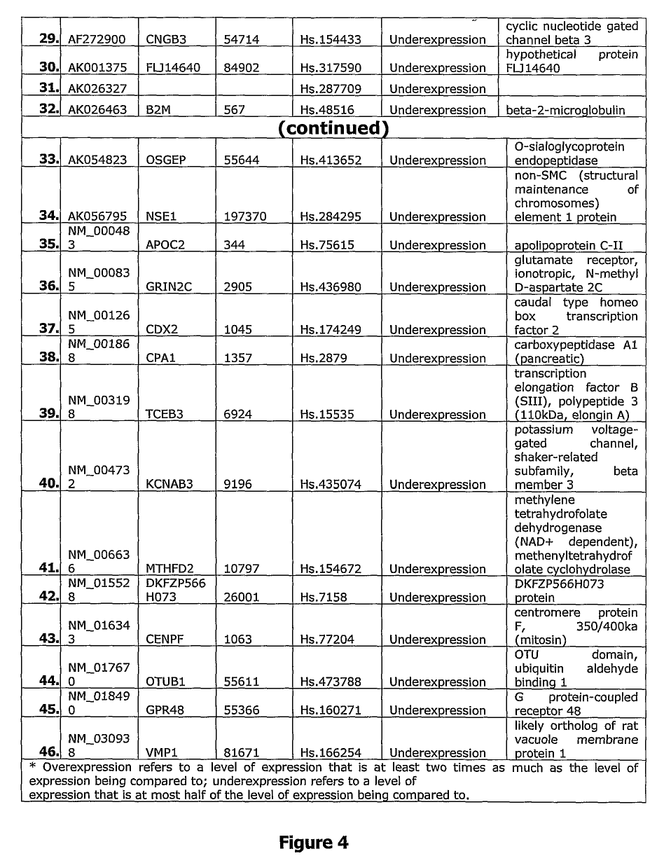

The invention also provides methods of detecting differential gene expression in intestinal gastric tissue in a mammal comprising (a) detecting levels of expression of at least two of the genes shown in FIG. 4 in an intestinal gastric tissue of the mammal, wherein the tissue is suspected of being cancerous; (b) detecting levels of expression of said genes in an intestinal gastric tissue adjacent to the tissue suspected of being cancerous of the mammal; and c) comparing the levels of expressions of said genes between the tissue suspected of being cancerous and the adjacent tissue. In some embodiments, the expression levels of at least 3, at least 4, at least 5, at least 10, at least 20, at least 30, at least 40, or 46 of genes from FIG. 4 are detected.

The methods described herein can each be carried out separately. Alternatively, the methods may be carried out simultaneously, i.e., they may share some common steps. For example, expression levels of the different subset of genes shown in FIGS. 1-4 in various tissues may be detected simultaneously. The expression levels of various genes can then be separately analyzed for differential expressions.

Intestinal gastric tissue samples can be obtained by standard techniques and may be stabilized in reagents such as Trizol prior to the analysis.

A tissue that is suspected of being cancerous (such as a tissue that is cancerous) can be identified by a clinician, using methods known in the art. A tissue may be suspected of being cancerous regardless of the health and/or disease status of the individual. For example, the individual may be a patient, a study participant, a screening subject, or any other class of individual from whom a sample is obtained and assessed in the context of the invention. The individual whose intestinal gastric tissue is suspected of being cancerous may be someone who is diagnosed with intestinal gastric cancer by other criteria, has one or more symptom of intestinal gastric cancer, or may have a predisposing factor, such as a genetic or medical history factor, for intestinal gastric cancer.

Differential expression of the genes can be detected at the mRNA levels and/or the protein levels. Total RNA and/or protein may be isolated using standard techniques known in the art. For example, methods for RNA isolation include those described in standard molecular biology textbooks. Commercially available kits such as those provided by Qiagen (RNeasy Kit) may also be used for RNA isolation. In some embodiments, the mRNA is converted to nucleic acid derived from the mRNA, for example, cDNA, and/or amplified, prior to detection of the expression levels.

Numerous methods for obtaining expression data are known, and any one or more of these techniques, singly or in combination, are suitable for determining gene expression in the context of the present invention. For example, expression patterns can be evaluated by northern analysis, PCR, RT-PCR, Taq Man analysis, FRET detection, monitoring one or more molecular beacon, hybridization to an oligonucleotide array, hybridization to a cDNA array, hybridization to a polynucleotide array, hybridization to a liquid microarray, hybridization to a microelectric array, molecular beacons, serial analysis of gene expression (SAGE), subtractive hybridization, differential display and/or differential screening (see, e.g., Lockhart and Winzeler (2000) Nature 405:827-836, and references cited therein).

For example, specific PCR primers are designed to a gene listed in FIGS. 1-4. cDNA is prepared from subject sample RNA by reverse transcription from a poly-dT oligonucleotide primer, and subjected to PCR. Double stranded cDNA may be prepared using primers suitable for reverse transcription of the PCR product, followed by amplification of the cDNA using in vitro transcription. The product of in vitro transcription is a sense-RNA corresponding to the gene. PCR product may also be evaluated in a number of ways known in the art, including real-time assessment using detection of labeled primers, e.g. TaqMan or molecular beacon probes. Technology platforms suitable for analysis of PCR products include the ABI 7700, 5700, or 7000 Sequence Detection Systems (Applied Biosystems, Foster City, Calif.), the MJ Research Opticon (MJ Research, Waltham, Mass.), the Roche Light Cycler (Roche Diagnostics, Indianapolis, Ind.), the Stratagene MX4000 (Stratagene, La Jolla, Calif.), and the Bio-Rad iCycler (Bio-Rad Laboratories, Hercules, Calif.). Alternatively, molecular beacons are used to detect presence of a nucleic acid sequence in an unamplified RNA or cDNA sample, or following amplification of the sequence using any method, e.g., IVT (in vitro transcription) or NASBA (nucleic acid sequence based amplification). Molecular beacons are designed with sequences complementary to a gene listed in FIGS. 1-4, and are linked to fluorescent labels. Each probe has a different fluorescent label with non-overlapping emission wavelengths. For example, expression of ten genes may be assessed using ten different sequence-specific molecular beacons.

Alternatively, or in addition, molecular beacons are used to assess expression of multiple nucleotide sequences simultaneously. Molecular beacons with sequences complimentary to two or more genes listed in FIGS. 1-4 are designed and linked to fluorescent labels. Each fluorescent label used must have a non-overlapping emission wavelength. For example, 10 nucleotide sequences can be assessed by hybridizing 10 sequence specific molecular beacons (each labeled with a different fluorescent molecule) to an amplified or non-amplified RNA or cDNA sample. Such an assay bypasses the need for sample labeling procedures.

Alternatively, or in addition, bead arrays can be used to assess expression of multiple sequences simultaneously (see, e.g., LabMAP 100, Luminex Corp, Austin, Tex.). Alternatively, or in addition, electric arrays can be used to assess expression of multiple sequences, as exemplified by the e-Sensor technology of Motorola (Chicago, Ill.) or Nanochip technology of Nanogen (San Diego, Calif.).

Of course, the particular method elected will be dependent on such factors as quantity of RNA recovered, practitioner preference, available reagents and equipment, detectors, and the like. Typically, however, the elected method(s) will be appropriate for processing the number of samples and probes of interest. Methods for high-throughput expression analysis are discussed below.

Alternatively, expression at the levels of protein products of gene expression is determined. For example, protein expression in a sample can be evaluated by one or more method selected from among: western analysis, two-dimensional gel analysis, chromatographic separation, mass spectrometric detection, protein-fusion reporter constructs, calorimetric assays, binding to a protein array (e.g., antibody array), and characterization of polysomal mRNA. One particularly favorable approach involves binding of labeled protein expression products to an array of antibodies specific for products of two or more genes listed in FIGS. 1-4. Methods for producing and evaluating antibodies are well known in the art, see, e.g., Coligan, supra; and Harlow and Lane (1989) Antibodies: A Laboratory Manual, Cold Spring Harbor Press, NY (“Harlow and Lane”). Additional details regarding a variety of immunological and immunoassay procedures adaptable to the present invention by selection of antibody reagents specific for the products of two or more genes listed in FIGS. 1-4 can be found in, e.g., Stites and Terr (eds.) (1991) Basic and Clinical Immunology, 7th ed. Another approach uses systems for performing desorption spectrometry. Commercially available systems, e.g., from Ciphergen Biosystems, Inc. (Fremont, Calif.) are particularly well suited to quantitative analysis of protein expression. Protein Chip® arrays (see, e.g., the website, ciphergen.com) used in desorption spectrometry approaches provide arrays for detection of protein expression. Alternatively, affinity reagents, (e.g., antibodies, small molecules, etc.) may be developed that recognize epitopes of one or more protein products. Affinity assays are used in protein array assays, e.g., to detect the presence or absence of particular proteins. Alternatively, affinity reagents are used to detect expression using the methods described above. In the case of a protein that is expressed on a cell surface, labeled affinity reagents are bound to a sample, and cells expressing the protein are identified and counted using fluorescent activated cell sorting (FACS).

A number of suitable high throughput formats exist for evaluating gene expression. Typically, the term high throughput refers to a format that performs at least about 100 assays, or at least about 500 assays, or at least about 1000 assays, or at least about 5000 assays, or at least about 10,000 assays, or more per day. When enumerating assays, either the number of samples or the number of candidate nucleotide sequences evaluated can be considered. For example, a northern analysis of, e.g., about 100 samples performed in a girded array, e.g., a dot blot, using a single probe corresponding to a gene described herein can be considered a high throughput assay. Alternatively, methods that simultaneously evaluate expression of about 100 or more genes in one or more samples, or in multiple samples, are considered high throughput.

Numerous technological platforms for performing high throughput expression analysis are known. Generally, such methods involve a logical or physical array of the subject samples. Common array formats include both liquid and solid phase arrays. For example, assays employing liquid phase arrays, e.g., for hybridization of nucleic acids, binding of antibodies or other receptors to ligand, etc., can be performed in multiwell, or microtiter, plates. Microtiter plates with 96, 384 or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis. Exemplary systems include, e.g., the ORCA™ system from Beckman-Coulter, Inc. (Fullerton, Calif.) and the Zymate systems from Zymark Corporation (Hopkinton, Mass.).

Alternatively, a variety of solid phase arrays can be employed to determine gene expression in the context of the invention. Exemplary formats include membrane or filter arrays (e.g., nitrocellulose, nylon), pin arrays, and bead arrays (e.g., in a liquid “slurry”). Typically, probes corresponding to nucleic acid or protein reagents that specifically interact with (e.g., hybridize to or bind to) an expression product corresponding to two or more gene shown in FIGS. 1-4, are immobilized, for example by direct or indirect cross-linking, to the solid support. Essentially any solid support capable of withstanding the reagents and conditions necessary for performing the particular expression assay can be utilized. For example, functionalized glass, silicon, silicon dioxide, modified silicon, any of a variety of polymers, such as (poly)tetrafluoroethylene, (poly)vinylidenedifluoride, polystyrene, polycarbonate, or combinations thereof can all serve as the substrate for a solid phase array.

In one embodiment, the array is a “chip” composed, e.g., of one of the above-specified materials. Polynucleotide probes, e.g., RNA or DNA, such as cDNA, synthetic oligonucleotides, and the like, or binding proteins such as antibodies or antigen-binding fragments or derivatives thereof, that specifically interact with expression products of genes listed in FIGS. 1-4 are affixed to the chip in a logically ordered manner, i.e., in an array. In addition, any molecule with a specific affinity for either the sense or anti-sense sequence of the nucleic acid sequence of genes listed in FIGS. 1-4 (depending on the design of the sample labeling), can be fixed to the array surface without loss of specific affinity for the marker and can be obtained and produced for array production, for example, proteins that specifically recognize the specific nucleic acid sequence of the genes, ribozymes, peptide nucleic acids (PNA), or other chemicals or molecules with specific affinity.

Detailed discussion of methods for linking nucleic acids and proteins to a chip substrate, are found in, e.g., U.S. Pat. No. 5,143,854, “Large Scale Photolithographic Solid Phase Synthesis Of Polypeptides And Receptor Binding Screening Thereof,” to Pirrung et al., issued, Sep. 1, 1992; U.S. Pat. No. 5,837,832, “Arrays Of Nucleic Acid Probes On Biological Chips,” to Chee et al., issued Nov. 17, 1998; U.S. Pat. No. 6,087,112, “Arrays With Modified Oligonucleotide And Polynucleotide Compositions,” to Dale, issued Jul. 11, 2000; U.S. Pat. No. 5,215,882, “Method Of Immobilizing Nucleic Acid On A Solid Substrate For Use In Nucleic Acid Hybridization Assays,” to Bahl et al., issued Jun. 1, 1993; U.S. Pat. No. 5,707,807, “Molecular Indexing For Expressed Gene Analysis,” to Kato, issued Jan. 13, 1998; U.S. Pat. No. 5,807,522, “Methods For Fabricating Microarrays Of Biological Samples,” to Brown et al., issued Sep. 15, 1998; U.S. Pat. No. 5,958,342, “Jet Droplet Device,” to Gamble et al., issued Sep. 28, 1999; U.S. Pat. No. 5,994,076, “Methods Of Assaying Differential Expression,” to Chenchik et al., issued Nov. 30, 1999; U.S. Pat. No. 6,004,755, “Quantitative Microarray Hybridization Assays,” to Wang, issued Dec. 21, 1999; U.S. Pat. No. 6,048,695, “Chemically Modified Nucleic Acids And Method For Coupling Nucleic Acids To Solid Support,” to Bradley et al., issued Apr. 11, 2000; U.S. Pat. No. 6,060,240, “Methods For Measuring Relative Amounts Of Nucleic Acids In A Complex Mixture And Retrieval Of Specific Sequences Therefrom,” to Kamb et al., issued May 9, 2000; U.S. Pat. No. 6,090,556, “Method For Quantitatively Determining The Expression Of A Gene,” to Kato, issued Jul. 18, 2000; and U.S. Pat. No. 6,040,138, “Expression Monitoring By Hybridization To High Density Oligonucleotide Arrays,” to Lockhart et al., issued Mar. 21, 2000.

For example, cDNA inserts corresponding to a gene listed in FIGS. 1-4, in a standard TA cloning vector, are amplified by a polymerase chain reaction for approximately 30-40 cycles. The amplified PCR products are then arrayed onto a glass support by any of a variety of well-known techniques, e.g., the VSLIPS™ technology described in U.S. Pat. No. 5,143,854. RNA, or cDNA corresponding to RNA, isolated from a subject sample, is labeled, e.g., with a fluorescent tag, and a solution containing the RNA (or cDNA) is incubated under conditions favorable for hybridization, with the “probe” chip. Following incubation, and washing to eliminate non-specific hybridization, the labeled nucleic acid bound to the chip is detected qualitatively or quantitatively, and the resulting expression profile for the corresponding gene is recorded. Multiple cDNAs from a nucleotide sequence that are non-overlapping or partially overlapping may also be used.

In another approach, oligonucleotides corresponding to two or more genes shown in FIGS. 1-4 are synthesized and spotted onto an array. Alternatively, oligonucleotides are synthesized onto the array using methods known in the art, e.g. Hughes, et al. supra. The oligonucleotide is designed to be complementary to any portion of the candidate nucleotide sequence. In addition, in the context of expression analysis for, e.g. diagnostic use of diagnostic nucleotide sets, an oligonucleotide can be designed to exhibit particular hybridization characteristics, or to exhibit a particular specificity and/or sensitivity, as further described below.

A hybridization signal may be amplified using methods known in the art, and as described herein, for example use of the Clontech kit (Glass Fluorescent Labeling Kit), Stratagene kit (Fairplay Microarray Labeling Kit), the Micromax kit (New England Nuclear, Inc.), the Genisphere kit (3DNA Submicro), linear amplification, e.g., as described in U.S. Pat. No. 6,132,997 or described in Hughes, T R, et al. (2001) Nature Biotechnology 19:343-347 (2001) and/or Westin et al. (2000) Nat Biotech. 18:199-204. In some cases, amplification techniques do not increase signal intensity, but allow assays to be done with small amounts of RNA.

Alternatively, fluorescently labeled cDNA are hybridized directly to the microarray using methods known in the art. For example, labeled cDNA are generated by reverse transcription using Cy3- and Cy5-conjugated deoxynucleotides, and the reaction products purified using standard methods. It is appreciated that the methods for signal amplification of expression data useful for identifying diagnostic nucleotide sets are also useful for amplification of expression data for diagnostic purposes.

Microarray expression may be detected by scanning the microarray with a variety of laser or CCD-based scanners, and extracting features with numerous software packages, for example, Imagene (Biodiscovery), Feature Extraction Software (Agilent), Scanalyze (Eisen, M. 1999. SCANALYZE User Manual; Stanford Univ., Stanford, Calif. Ver 2.32.), GenePix (Axon Instruments).

In another approach, hybridization to microelectric arrays is performed, e.g., as described in Umek et al (2001) J Mol Diagn. 3:74-84. An affinity probe, e.g., DNA, is deposited on a metal surface. The metal surface underlying each probe is connected to a metal wire and electrical signal detection system. Unlabelled RNA or cDNA is hybridized to the array, or alternatively, RNA or cDNA sample is amplified before hybridization, e.g., by PCR. Specific hybridization of sample RNA or cDNA results in generation of an electrical signal, which is transmitted to a detector. See Westin (2000) Nat Bio tech. 18:199-204 (describing anchored multiplex amplification of a microelectronic chip array); Edman (1997) NAR 25:4907-14; Vignali (2000) J Immunol Methods 243:243-55.

Expression patterns can be evaluated by qualitative and/or quantitative measures. Certain of the above described techniques for evaluating gene expression (e.g., as RNA or protein products) yield data that are predominantly qualitative in nature, i.e., the methods detect differences in expression that classify expression into distinct modes without providing significant information regarding quantitative aspects of expression. For example, a technique can be described as a qualitative technique if it detects the presence or absence of expression of a candidate nucleotide sequence, i.e., an on/off pattern of expression. Alternatively, a qualitative technique measures the presence (and/or absence) of different alleles, or variants, of a gene product.

In contrast, some methods provide data that characterize expression in a quantitative manner. That is, the methods relate expression on a numerical scale, e.g., a scale of 0-5, a scale of 1-10, a scale of +−+++, from grade 1 to grade 5, a grade from a to z, or the like. It will be understood that the numerical, and symbolic examples provided are arbitrary, and that any graduated scale (or any symbolic representation of a graduated scale) can be employed in the context of the present invention to describe quantitative differences in nucleotide sequence expression. Typically, such methods yield information corresponding to a relative increase or decrease in expression. Any method that yields either quantitative or qualitative expression data is suitable for evaluating expression of the genes. In some cases, e.g., when multiple methods are employed to determine expression patterns for a plurality of candidate nucleotide sequences, the recovered data, e.g., the expression profile, for the nucleotide sequences is a combination of quantitative and qualitative data.

In some applications, expressions of a plurality of genes are evaluated sequentially. This is typically the case for methods that can be characterized as low- to moderate throughput. In contrast, as the throughput of the elected assay increases, expression for the plurality of genes in a sample or multiple samples is typically assayed simultaneously. Again, the methods (and throughput) are largely determined by the individual practitioner, although, typically, it is preferable to employ methods that permit rapid, e.g. automated or partially automated, preparation and detection, on a scale that is time-efficient and cost-effective.

In some embodiments, the comparison of genes expression levels is carried out by first establishing an expression profile by a statistical algorithm that determines the optimal relation between patterns of expression of various genes and then comparing the expression profiles of the two tissue samples to be compared.

### Method of Diagnosing Intestinal Gastric Cancer

The expression levels of genes described herein can be used as a basis for diagnosing or determining susceptibility of a mammal to intestinal gastric cancer. Diagnosis includes, for example, determining presence or absence of intestinal gastric cancer or a symptom of intestinal gastric cancer in a mammal who has, who is suspected of having, or who may be suspected of being predisposed to an intestinal gastric cancer. Diagnosis may also include providing a preliminary basis for further determination (or confirmation) of the presence of absence or intestinal gastric cancer by other methods. In one aspect, the invention provides a method for diagnosing intestinal gastric cancer in a mammal, comprising (a) detecting levels of expression of at least two genes shown in FIG. 1 in an intestinal gastric tissue in a mammal, wherein the tissue is suspected of being cancerous; (b) detecting levels of expression of said genes in an intestinal gastric tissue adjacent to the tissue suspected of being cancerous of the mammal; and (c) comparing the levels of expression of said genes of the tissue suspected of being cancerous and the adjacent tissue, wherein substantial variance of the levels of expression of at least two gene in FIG. 1 between the tissue suspected of being cancerous and the adjacent tissue is indicative of the presence of intestinal gastric cancer in the mammal.

In some embodiments, the expression levels of at least 2, at least 3, at least at least 5, at least 10, at least 20, at least 30, at least 40, at least 50, at least 60, at least 70, at least 80, at least 90, at least 100, or 102 of genes shown in FIG. 1 are detected, wherein substantial variance of at least 60% of the genes between the tissue suspected of being cancerous and the adjacent tissue is indicative of the presence of intestinal gastric cancer. In some embodiments, the expression levels of at least one of genes 1-71 in FIG. 1 and at least one of genes 72-102 in FIG. 1 are detected, wherein substantially decreased expression of at least one of genes 1-71 in FIG. 1 and substantially increased expression of at least one of genes 72-102 in FIG. 1 in the tissue suspected of being cancerous as compared to the adjacent tissue are indicative of the presence of intestinal gastric cancer in the mammal. In some embodiments, the levels of expression of all the genes in FIG. 1 are detected, wherein substantially decreased expression of at least one of genes 1-71 in FIG. 1 and substantially increased expression of at least one of genes 72-102 in FIG. 1 in the tissue suspected of being cancerous as compared to the adjacent tissue are indicative of presence of intestinal gastric cancer in the mammal.

In some embodiments, the levels of expression of all the genes in FIG. 1 are detected, wherein substantially decreased expression of at least 2, at least 5, at least 10, at least 20, at least 30, at least 40, at least 42, at least 50, at least 60, or at least 70 of genes 1-71 in FIG. 1 and/or substantially increased expression of at least 2, at least 5, at least 10, at least 18, at least 20, or at least 30 of genes 72-102 in FIG. 1 in the tissue suspected of being cancerous as compared to the adjacent tissue are indicative of presence of intestinal gastric cancer in the mammal.

In some embodiments, the levels of expressions of all the genes in FIG. 1 are detected, wherein substantially decreased expression of genes 1-71 in FIG. 1 and/or substantially increased expression of genes 72-102 in FIG. 1 in the tissue suspected of being cancerous as compared to the adjacent tissue are indicative of presence of intestinal gastric cancer in the mammal.

In another aspect, the invention provides a method for diagnosing intestinal gastric cancer in a mammal by detecting differential expression of genes shown in FIG. 2. In some embodiments, the method comprises (a) detecting levels of expression of at least two genes shown in FIG. 2 in an intestinal gastric tissue of a mammal, wherein the tissue is suspected of being cancerous; (b) comparing the levels of expression of said genes between the tissue suspected of being cancerous and a normal intestinal gastric mucosa tissue of a mammal of the same species, wherein substantial variance of the levels of expression of at least two genes shown in FIG. 2 between the gastric tissue suspected of being cancerous and the normal gastric mucosa tissue is indicative of presence of intestinal gastric cancer in the individual. In some embodiments, the method comprises (a) detecting levels of expression of at least two genes shown in FIG. 2 in an intestinal gastric tissue adjacent to an intestinal gastric tissue suspected of being cancerous, and (b) comparing the levels of expressions of said genes between the adjacent tissue and a normal intestinal gastric mucosa tissue of an individual of the same species, wherein substantial variance of the levels of expression of at least two genes shown in FIG. 2 between the adjacent tissue and the normal gastric mucosa tissue is indicative of presence of intestinal gastric cancer in the individual.

In some embodiments, the method comprises: (a) detecting levels of expression of at least two genes shown in FIG. 2 in an intestinal gastric tissue of a mammal (such as a mammal), wherein the tissue is suspected of being cancerous; (b) detecting levels of expression of said genes in an intestinal gastric tissue adjacent to the tissue suspected of being cancerous of the individual, and (c) comparing the levels of expressions of said genes between the tissue suspected of being cancerous (and/or the adjacent tissue) with that of a normal intestinal gastric mucosa tissue of a mammal of the same species; and (d) comparing the levels of expression of the genes in the gastric tissue suspected of being cancerous to the levels of expression of the genes in the adjacent gastric tissue; wherein substantial variance of the levels of expression of the genes shown in FIG. 2 between the gastric tissue suspected of being cancerous (and/or the adjacent gastric tissue) and the normal gastric mucosa tissue, and no substantial variance of the levels of expression of the genes shown in FIG. 2 between the gastric tissue suspected of being cancerous and the adjacent gastric tissue are indicative of presence of intestinal gastric cancer in the mammal.

In some embodiments, the method comprises (a) detecting levels of expression of at least two genes shown in FIG. 2 in an intestinal gastric tissue suspected of being cancerous of the mammal; (b) detecting levels of expression of the genes in an intestinal gastric tissue adjacent to the gastric tissue suspected of being cancerous; (c) comparing the levels of expression of the genes in the gastric tissue suspected of being cancerous to levels of expression of the genes in a normal intestinal gastric mucosa tissue of the same mammal species; (d) comparing the levels of expression of the genes in the adjacent gastric tissue to levels of expression of the genes in a normal intestinal gastric mucosa tissue of the same mammal species; and (e) comparing the levels of expression of the genes in the gastric tissue suspected of being cancerous to the levels of expression of the genes in the adjacent gastric tissue; wherein substantial variance of the levels of expression of the genes shown in FIG. 2 between the gastric tissue suspected of being cancerous and the normal gastric mucosa tissue and between the adjacent gastric tissue and the normal gastric mucosa tissue, and no substantial variance of the levels of expression of the genes shown in FIG. 2 between the gastric tissue suspected of being cancerous and the adjacent gastric tissue are indicative of presence of intestinal gastric cancer in the mammal.

In some embodiments, the expression levels of at least 2, at least 3, at least at least 5, at least 10, at least 20, at least 30, at least 40, at least 50, at least 60, at least 70, at least 80, or 84 of genes from FIG. 2 are detected, wherein substantial variance of the levels expression of at least about 60% of the genes between the tissue suspected of being cancerous (and/or the adjacent tissue) and the normal tissue is indicative of the presence of intestinal gastric cancer. In some embodiments, at least one of genes 1-53 in FIG. 2 and at least one of genes 54-84 in FIG. 2 are detected, wherein substantially decreased expression of at least one of genes 1-53 in FIG. 2 and substantially increased expression of at least one of genes 54-84 in FIG. 2 in the tissue suspected of being cancerous (and/or the adjacent tissue) as compared to the normal tissue are indicative of the presence of intestinal gastric cancer in the mammal. In some embodiments, the levels of expressions of all the genes in FIG. 2 are detected, wherein substantially decreased expression of at least one of genes 1-53 in FIG. 2 and substantially increased expression of at least one of genes 54-84 in FIG. 2 in the tissue suspected of being cancerous (and/or the adjacent tissue) as compared to the normal tissue are indicative of presence of intestinal gastric cancer in the mammal.

In some embodiments, the levels of expressions of all the genes in FIG. 2 are detected, wherein substantially decreased expression of at least 2, at least 5, at least 10, at least 20, at least 30, at least 31, at least 40, or at least 50 of genes 1-53 in FIG. 2 and/or substantially increased expression of at least 2, at least 5, at least 10, at least 18, at least 20, or at least 30 of genes 54-84 in FIG. 2 in the tissue suspected of being cancerous (and/or the adjacent tissue) as compared to the normal tissue are indicative of presence of intestinal gastric cancer in the mammal.

In some embodiments, the levels of expressions of all the genes in FIG. 2 are detected, wherein substantially decreased expression of genes 1-53 in FIG. 2 and/or substantially increased expression of genes 54-84 in FIG. 2 in the tissue suspected of being cancerous (and/or the adjacent tissue) as compared to the normal tissue are indicative of presence of intestinal gastric cancer in the mammal.

In some embodiments, qualitative and/or quantitative levels of gene expression in a tissue sample are compared with levels of expression in a reference expression profile that are indicative of the presence or absence of intestinal gastric cancer or condition(s) associated with intestinal gastric cancer. To obtain a diagnosis, the levels of gene expression in a sample may be compared to one or more than one expression profile, each of which may be indicative of presence or absence of intestinal gastric cancer.

Accordingly, in some embodiments, the method comprises (a) detecting levels of expression of at least two genes shown in FIG. 2 in an intestinal gastric tissue of an individual to obtain an expression profile of the detected genes, wherein the tissue is suspected of being cancerous; and (b) comparing the expression profile of the individual with that of a normal intestinal gastric mucosa tissue of an individual of the same species, wherein a substantial difference in the expression profiles are indicative of presence of intestinal gastric cancer in the individual. In some embodiments, the method comprises (a) detecting levels of expression of at least two genes shown in FIG. 2 in an intestinal gastric tissue of an individual to obtain an expression profile of the detected genes, wherein the tissue is suspected of being cancerous; and (b) comparing the expression profile of the individual with an expression profile of a intestinal gastric cancer tissue from another individual of the same species, wherein a substantial similarity in the expression profiles are indicative of presence of intestinal gastric cancer in the individual. In some embodiments, the expression profiles of an intestinal gastric tissue adjacent to the tissue suspected of being cancerous (instead of the intestinal gastric tissue suspected of being cancerous) is used for comparison.

In some embodiments, the invention provides a method of determining the extent of progression of intestinal gastric cancer in an individual. For example, qualitative and/or quantitative expression data from an intestinal gastric tissue that cancerous or suspected of being cancerous (and/or the adjacent tissue) can be compared with a reference expression profile that are indicative of the extent of progression of intestinal gastric cancer. The reference expression profile may be from another individual with the same or a different stage of intestinal gastric cancer or may be established from a compilation of data from multiple individuals.

In some embodiments, polynucleotides derived from a sample from an individual (e.g., mRNA or polynucleotides derived from mRNA, for example cDNA) are contacted with isolated polynucleotide molecules in a system for detecting gene expression as described further herein, wherein each isolated polynucleotide molecule detects an expressed product of a gene shown in FIG. 1 and/or FIG. 2, and hybridization complexes formed, if any, are detected, wherein the presence, absence, or amount of hybridization complexes formed from at least one of the isolated polynucleotides are indicative of presence or absence of intestinal gastric cancer in the individual. In some embodiments, presence, absence, or amount of the polynucleotides derived from the sample is compared with the presence, absence, or amount of polynucleotides in an expression profile indicative of presence or absence of intestinal gastric cancer.

In some embodiments, polypeptides derived from a sample from an individual are contacted with a system for detecting gene expression as described further herein which comprises molecules capable of detectably binding to polypeptides that are differentially expressed in intestinal gastric cancer, for example, antibodies or antigen binding fragments thereof, that detect expressed polypeptide products of genes corresponding to polynucleotide sequences listed in FIG. 1 and/or FIG. 2, wherein the presence, absence, or amount of bound polypeptide are indicative of presence or absence, or amount of polypeptides derived from the sample is compared with presence, absence, or amount of polypeptides in an expression profile indicative of presence or absence of intestinal gastric cancer.

The methods described herein may independently provide indicia of the presence or absence of intestinal gastric cancer. Alternatively, they may be carried out in various combinations to determine the presence or absence of intestinal gastric cancer. The methods may also be carried out in combination with other methods (such as methods known in the art) to determine the presence or absence of intestinal gastric cancer. In some embodiments, the methods may serve to confirm the presence or absence of intestinal gastric cancer diagnosed by other methods (such as methods known in the art or other methods described herein).

The methods described herein may also be used for determining prognosis of intestinal gastric cancer in an individual or determining susceptibility of the individual to intestinal gastric cancer. “Prognosis” as used herein refers to the probability that an individual will develop an intestinal gastric cancer symptom or condition, or that intestinal gastric cancer will progress in an individual who has intestinal gastric cancer. Prognosis is a determination or prediction of probable course and/or outcome of a disease condition, i.e., whether an individual will exhibit or develop symptoms of the disease, i.e., clinical event.

The methods may also be useful for monitoring the progression of intestinal gastric cancer in a mammal. The methods may also be useful for risk stratification, assessing suitability of the individual for a particular treatment, predicting effectiveness of a treatment or treatment outcome for an individual, or providing information relating to an individual's health status.

### Methods of Assessing Extent of Differentiation of Intestinal Gastric Cancer

In another aspect, the invention provides methods of assessing levels of differentiation of intestinal gastric cancer in a mammal, such as in a patient who has been diagnosed with intestinal gastric cancer by methods known in the art or by methods described herein.

In some embodiments, the invention provides a method for assessing levels of differentiation of intestinal gastric cancer in a mammal, comprising (a) detecting levels of expression of at least two genes shown in FIG. 3 in an intestinal gastric cancer tissue of the mammal, (b) detecting levels of expression of said genes in an intestinal gastric tissue adjacent to the cancer tissue, and (c) comparing the levels of expression of said genes between the cancer tissue and the adjacent tissue, wherein substantial variance of the levels of expression of at least two genes between the cancer tissue and the adjacent tissue is indicative of high level of differentiation in the intestinal gastric cancer tissue. Because the comparison is carried between different tissues from the same individual, gene expression variance in the population may be avoided. The invention thus provides individualized method for assessing levels of differentiation.

In some embodiments, the expression levels of at least 2, at least 3, at least 5, at least 10, at least 15, at least 20, at least 25, at least 30, at least 35, at least 40, at least 45, at least 50, or 55 of genes shown in FIG. 3 are detected, wherein substantial variance of at least 60% of the genes between the cancer tissue and the adjacent tissue is indicative of high level of differentiation in the intestinal gastric cancer tissue. In some embodiments, the expression levels of at least one of genes 1-16 shown in FIG. 3 and at least one of genes 17-55 shown in FIG. 3 are detected, wherein substantially increased expression of at least one of genes 1-16 shown in FIG. 3 and substantially decreased expression of at least one of genes 17-55 shown in FIG. 3 in the cancer tissue as compared to the adjacent tissue are indicative of high level of differentiation in the intestinal gastric cancer tissue.

In some embodiments, the expression levels of all genes shown in FIG. 3 are detected, wherein substantially increased expression of at least one of genes 1-16 shown in FIG. 3 and substantially decreased expression of at least one of genes 17-55 shown in FIG. 3 in the cancer tissue as compared to the adjacent tissue are indicative of high level of differentiation in the intestinal gastric cancer tissue. In some embodiments, the expression levels of all genes shown in FIG. 3 are detected, wherein substantially increased expression of at least 2, at least 3, at least 5, at least 9, at least 10, or at least 15 of genes 1-16 in FIG. 3 and/or substantially decreased expression of at least 2, at least 3, at least 5, at least 10, at least 15, at least 20, at least 23, at least 25, at least 30, or at least 35 of genes 17-55 in FIG. 3 in the cancer tissue as compared to the adjacent tissue are indicative of high level of differentiation in the intestinal gastric cancer tissue. In some embodiments, all genes shown in FIG. 3 are detected, wherein substantially increased expression of genes 1-16 in FIG. 3 and/or substantially decreased expression of genes 17-55 in FIG. 3 in the cancer tissue as compared to the adjacent tissue are indicative of high level of differentiation in the intestinal gastric cancer tissue.

In another aspect, the invention provides a method for assessing levels of differentiation of intestinal gastric cancer in a mammal, comprising (a) detecting levels of expression of at least two genes shown in FIG. 4 in an intestinal gastric cancer tissue of the mammal, (b) detecting levels of expression of said genes in an intestinal gastric tissue adjacent to the cancer tissue, and (c) comparing the levels of expression of said genes between the cancer tissue and the adjacent tissue, wherein substantial variance of the levels of expression of at least two genes between the cancer tissue and the adjacent tissue is indicative of low level of differentiation in the intestinal gastric cancer tissue.

In some embodiments, the expression levels of at least 2, at least 3, at least 5, at least 10, at least 15, at least 20, at least 25, at least 30, at least 35, at least 40, or 46 of genes shown in FIG. 4 are detected, wherein substantial variance of at least 60% of the genes between the cancer tissue and the adjacent tissue is indicative of low level of differentiation in the intestinal gastric cancer tissue. In some embodiments, the expression levels of at least one of genes 1-28 shown in FIG. 4 and at least one of genes 29-46 shown in FIG. 4 are detected, wherein substantially increased expression of at least one of genes 1-28 in FIG. 4 and substantially decreased expression of at least one of genes 29-46 in FIG. 4 in the cancer tissue as compared to the adjacent tissue are indicative of low level of differentiation in the intestinal gastric cancer tissue.

In some embodiments, the expression levels of all genes shown in FIG. 4 are detected, wherein substantial increase in expression of at least one of genes 1-28 in FIG. 4 and substantially decreased expression of at least one of genes 29-46 shown in FIG. 4 in the cancer tissue as compared to the adjacent tissue are indicative of low level of differentiation in the intestinal gastric cancer tissue. In some embodiments, the expression levels of all genes shown in FIG. 4 are detected, wherein substantially increased expression of at least 2, at least 3, at least 5, at least 10, at least 15, at least 16, at least 20, or at least 25 of genes 1-28 in FIG. 4 and/or substantially decreased expression of at least 2, at least 3, at least 5, at least 10, at least 11, or at least 15 of genes 29-46 in FIG. 4 in the cancer tissue as compared to the adjacent tissue are indicative of low level of differentiation in the intestinal gastric cancer tissue. In some embodiments, the expression levels of all genes shown in FIG. 4 are detected, wherein substantially increased expression of genes 1-28 in FIG. 4 and/or substantially decreased expression of genes 29-46 in FIG. 4 in the cancer tissue as compared to the adjacent tissue are indicative of low level of differentiation in the intestinal gastric cancer tissue.

In some embodiments, qualitative and/or quantitative levels of gene expression in a test sample are compared with levels of expression in an expression profile that are indicative of the levels of differentiation of intestinal gastric cancer. The levels of gene expression may be compared to one or more than one expression profile, each of which may be indicative of a different levels of differentiation of intestinal gastric cancer.

In some embodiments, polynucleotides derived from a sample from an individual (e.g., mRNA or polynucleotide derived from mRNA, for example cDNA) are contacted with isolated polynucleotide molecules that detect expressed polypeptide products of genes listed in FIG. 3 and/or FIG. 4 in a system for detecting gene expression as described herein, wherein each isolated polynucleotide molecule is capable of detecting an expressed product of a gene that is differentially expressed in intestinal gastric cancer in a mammal, and hybridization complexes formed, if any, are detected wherein presence, absence, or amount of hybridization complexes formed from at least two of the isolated polynucleotides are indicative of the levels of differentiation of an intestinal gastric cancer in the individual.

In some embodiments, polypeptides derived from a sample from an individual are contacted with a system for detecting gene expression as described herein which comprises molecules capable of detectably binding to polypeptide molecules that are differentially expressed in intestinal gastric cancer, for example, antibodies or antigen binding fragments thereof, that detect expressed polypeptide products of genes listed in FIG. 3 and/or FIG. 4, wherein presence, absence, or amount of bound polypeptide are indicative of levels of differentiation of intestinal gastric cancer in the individual.

### System for Detecting Gene Expression

The invention also provides systems for detecting expression of genes that are differentially expressed in intestinal gastric cancer tissue samples. The systems can be used for diagnosing intestinal gastric cancer and/or assessing levels of differentiation of intestinal gastric cancer.

In some embodiments, the system consists essentially of at least two isolated molecules, wherein each isolated molecule is capable of detecting expression of a different gene, wherein each gene is selected from the group consisting of genes 1-102 shown in FIG. 1. In some embodiments, the system consists essentially of at least 3, at least 5, at least 10, at least 20, at least 30, at least 40, at least 50, at least 60, at least 70, at least 80, at least 90, at least 100, or at least 102 isolated molecules, wherein each isolated molecule is capable of detecting expression of a different gene, wherein each gene is selected from the group consisting of genes 1-102 shown in FIG. 1. In some embodiments, the system consists essentially of a plurality of isolated molecules, wherein each isolated molecule is capable of detecting expression of a gene selected from the group consisting of at least 61, at least 70, at least 80, at least 90, at least 100, or 102 genes shown in FIG. 1, whereby differential expression of said genes shown in FIG. 1 can be detected.

In some embodiments, the system consists essentially of at least two isolated molecules, wherein each isolated molecule is capable of detecting expression of a different gene, wherein each gene is selected from the group consisting of genes 1-84 shown in FIG. 2. In some embodiments, the system consists essentially of at least 3, at least 5, at least 10, at least 20, at least 30, at least 40, at least 50, at least 60, at least 70, at least 80, or at least 84 isolated molecules, wherein each isolated molecule is capable of detecting expression of a different genes, wherein the gene is selected from the group consisting of genes 1-84 shown in FIG. 2. In some embodiments, the system consists essentially of a plurality of isolated molecules, wherein each isolated molecule is capable of detecting expression of a gene selected from the group consisting of at least 50, at least 60, at least 70, at least 80, or 84 genes shown in FIG. 2, whereby differential expression of said genes can be detected.

In some embodiments, the system consists essentially of at least two isolated molecules, wherein each isolated molecule is capable of detecting expression of a different gene, wherein each gene is selected from the group consisting of genes 1-55 shown in FIG. 3. In some embodiments, the system consists essentially of at least 3, at least 5, at least 10, at least 20, at least 30, at least 35, at least 40, at least 45, at least 50, or at least 55 isolated molecules, wherein each isolated molecule is capable of detecting expression of a different gene, wherein each gene is selected from the group consisting of genes 1-55 shown in FIG. 3. In some embodiments, the system consists essentially of a plurality of isolated molecules, wherein each isolated molecule is capable of detecting expression of a gene selected from the group consisting of at least 33, at least 40, at least 50, or 55 genes shown in FIG. 3, whereby differential expression of said genes can be detected.

In some embodiments, the system consists essentially of at least two isolated molecules, wherein each isolated molecule is capable of detecting expression of a different gene, wherein each gene is selected from the group consisting of genes 1-46 shown in FIG. 4. In some embodiments, the system consists essentially of at least 3, at least 5, at least 10, at least 20, at least 30, at least 35, at least 40, at least 45, or at least 46 isolated molecules, wherein each isolated molecule is capable of detecting expression of a different gene, wherein each gene is selected from the group consisting of genes 1-46 shown in FIG. 4. In some embodiments, the system consists essentially of a plurality of isolated molecules, wherein each isolated molecule is capable of detecting expression of a gene selected from the group consisting of at least 27, at least 30, at least 40, or 46 genes shown in FIG. 4, whereby differential expression of said genes can be detected.

Isolated molecules for detecting genes listed in FIGS. 1-4 may exist in a system for detecting differential gene expressions in various combinations. For example, in some embodiments, the system consists essentially of a plurality of (such as at least any of 2, 3, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 293) isolated molecules, each detecting an expression product of a different gene selected from the group consisting of genes shown in FIGS. 1-4. More than one isolated molecules may be in the system for detecting one gene. In some embodiments, the system consists essentially of isolated molecules, wherein each isolated molecule is capable of detecting expression of a gene selected from the group consisting of genes shown in FIGS. 1-4, whereby differential expression of genes shown in FIGS. 1-4 can be detected.

In some embodiments, the systems described herein comprise isolated polynucleotide molecules. It is understood that, for detection of gene expression, certain sequence variations are acceptable. Thus, the sequences of the isolated polynucleotides (or their complementary sequences) may be slightly different from those of the genes identified herein. Such sequence variations are understood to those of ordinary skill in the art to be variations in the sequence that do not significantly affect the ability of the sequences to detect gene expression. For example, homologs and variants of the genes disclosed herein may be used in the systems of the present invention. Homologs and variants of these polynucleotide molecules possess a relatively high degree of sequence identity when aligned using standard methods. Polynucleotide sequences encompassed by the invention have at least 40-50, 50-60, 70-80, 80-85, 85-90, 90-95 or 95-100% sequence identity to the sequence of the genes disclosed herein.

The degree of sequence identity required to detect gene expression varies depending on the length of an oligonucleotide. For example, for a 60mer (i.e., an oligonucleotide with 60 nucleotides), 6-8 random mutations or 6-8 random deletions do not affect gene expression detection. Hughes, T. R., et al. (2001) Nature Biotechnology 19:343-347. As the length of the polynucleotide sequence is increased, the number of mutations or deletions permitted while still allowing gene expression detection is increased.

In some embodiments, the isolated polynucleotide molecules are less than about any of the following lengths (in bases or base pairs): 10,000; 5000; 2500; 2000; 1500; 1250; 1000; 750; 500; 300; 250; 200; 175; 150; 125; 100; 75; 50; 25; 10. In some embodiments, isolated polynucleotide molecules are greater than about any of the following lengths (in bases or base pairs): 10; 15; 20; 25; 30; 40; 50; 60; 75; 100; 125; 150; 175; 200; 250; 300; 350; 400; 500; 750; 1000; 2000; 5000; 7500; 10,000; 20,000; 50,000. Alternately, an isolated polynucleotide molecule can be any of a range of sizes having an upper limit of 10,000; 5000; 2500; 2000; 1500; 1250; 1000; 750; 500; 300; 250; 200; 175; 150; 125; 100; 75; 50; 25; or 10 and an independently selected lower limit of 10; 15; 20; 25; 30; 40; 50; 60; 75; 100; 125; 150; 175; 200; 250; 300; 350; 400; 500; 750; 1000; 2000; 5000; or 7500, wherein the lower limit is less than the upper limit.

The isolated polynucleotides of the system for detecting gene expression may include DNA or RNA or a combination thereof, and/or modified forms thereof, and/or may also include a modified polynucleotide backbone. In some embodiments, the isolated polynucleotides are selected from the group consisting of synthetic oligonucleotides, genomic DNA, cDNA, RNA, or PNA.

In some embodiments, the systems described herein comprise molecules that are capable of detectably binding to polypeptides, including, but not limited to, antibodies or antigen binding fragments thereof.

In some embodiments, a system for detecting gene expression in accordance with the present invention is in the form of an array. “Microarray” and “array,” as used interchangeably herein, comprise a surface with an array, preferably ordered array, of putative binding (e.g., by hybridization) sites for a biochemical sample (target) which often has undetermined characteristics. In one embodiment, a microarray refers to an assembly of distinct polynucleotide or oligonucleotide probes immobilized at defined positions on a substrate. Arrays may be formed on substrates fabricated with materials such as paper, glass, plastic (e.g., polypropylene, nylon, polystyrene), polyacrylamide, nitrocellulose, silicon, optical fiber or any other suitable solid or semi-solid support, and configured in a planar (e.g., glass plates, silicon chips) or three-dimensional (e.g., pins, fibers, beads, particles, microtiter wells, capillaries) configuration. Probes forming the arrays may be attached to the substrate by any number of ways including (i) in situ synthesis (e.g., high-density oligonucleotide arrays) using photolithographic techniques (see, Fodor et al., Science (1991), 251:767-773; Pease et al., Proc. Natl. Acad. Sci. U.S.A. (1994), 91:5022-5026; Lockhart et al., Nature Biotechnology (1996), 14:1675; U.S. Pat. Nos. 5,578,832; 5,556,752; and 5,510,270); (ii) spotting/printing at medium to low-density (e.g., cDNA probes) on glass, nylon or nitrocellulose (Schena et al, Science (1995), 270:467-470, DeRisi et al, Nature Genetics (1996), 14:457-460; Shalon et al., Genome Res. (1996), 6:639-645; and Schena et al., Proc. Natl. Acad. Sci. U.S.A. (1995), 93:10539-11286); (iii) by masking (Maskos and Southern, Nuc. Acids. Res. (1992), 20:1679-1684) and (iv) by dot-blotting on a nylon or nitrocellulose hybridization membrane (see, e.g., Sambrook et al., Eds., 1989, Molecular Cloning: A Laboratory Manual, 2nd ed., Vol. 1-4, Cold Spring Harbor Laboratory (Cold Spring Harbor, N.Y.)). Probes may also be noncovalently immobilized on the substrate by hybridization to anchors, by means of magnetic beads, or in a fluid phase such as in microtiter wells or capillaries. The probe molecules are generally nucleic acids such as DNA, RNA, PNA, and cDNA but may also include proteins, polypeptides, oligosaccharides, cells, tissues and any permutations thereof which can specifically bind the target molecules.

For example, microarrays, in which either defined cDNAs or oligonucleotides are immobilized at discrete locations on, for example, solid or semi-solid substrates, or on defined particles, enable the detection and/or quantification of the expression of a multitude of genes in a given specimen.

Several techniques are well-known in the art for attaching nucleic acids to a solid substrate such as a glass slide. One method is to incorporate modified bases or analogs that contain a moiety that is capable of attachment to a solid substrate, such as an amine group, a derivative of an amine group or another group with a positive charge, into the amplified nucleic acids. The amplified product is then contacted with a solid substrate, such as a glass slide, which is coated with an aldehyde or another reactive group which will form a covalent link with the reactive group that is on the amplified product and become covalently attached to the glass slide. Microarrays comprising the amplified products can be fabricated using a Biodot (BioDot, Inc. Irvine, Calif.) spotting apparatus and aldehyde-coated glass slides (CEL Associates, Houston, Tex.). Amplification products can be spotted onto the aldehyde-coated slides, and processed according to published procedures (Schena et al., Proc. Natl. Acad. Sci. U.S.A. (1995) 93:10614-10619). Arrays can also be printed by robotics onto glass, nylon (Ramsay, G., Nature Biotechnol. (1998), 16:40-44), polypropylene (Matson, et al., Anal Biochem. (1995), 224(1):110-6), and silicone slides (Marshall, A. and Hodgson, J., Nature Biotechnol. (1998), 16:27-31). Other approaches to array assembly include fine micropipetting within electric fields (Marshall and Hodgson, supra), and spotting the polynucleotides directly onto positively coated plates. Methods such as those using amino propyl silicon surface chemistry are also known in the art, as disclosed at www.cmt.corning.com and http://cmgm.stanford.edu/pbrown/.

One method for making microarrays is by making high-density polynucleotide arrays. Techniques are known for rapid deposition of polynucleotides (Blanchard et al., Biosensors & Bioelectronics, 11:687-690). Other methods for making microarrays, e.g., by masking (Maskos and Southern, Nuc. Acids. Res. (1992), 20:1679-1684), may also be used. In principle, and as noted above, any type of array, for example, dot blots on a nylon hybridization membrane, could be used. However, as will be recognized by those skilled in the art, very small arrays will frequently be preferred because hybridization volumes will be smaller.

In one embodiment, the invention provides an array consisting essentially of at least two isolated polynucleotide molecules, wherein each isolated polynucleotide molecule is capable of detecting an expressed gene product of a gene selected from the group consisting of genes listed in FIG. 1. In one embodiment, the invention provides an array consisting essentially of at least two isolated polynucleotide molecules, wherein each isolated polynucleotide molecule is capable of detecting an expressed gene product of a gene selected from the group consisting of genes shown in FIG. 2. In one embodiment, the invention provides an array consisting essentially of at least two isolated polynucleotide molecules, wherein each isolated polynucleotide molecule is capable of detecting an expressed gene product of a gene selected from the group of genes shown in FIG. 3. In one embodiment, the invention provides an array consisting essentially of at least two isolated polynucleotide molecules, wherein each isolated polynucleotide molecule is capable of detecting an expressed gene product of a gene selected from the group of genes shown in FIG. 4. In various embodiments, an array in accordance with the invention comprises at least any of 2, 3, 5, 10, 15, 20, 25, 30, 40, 50, 60, 70, 80, 90, 100, 110, 120, 130, 140, 150, 160, 170, 180, 190, 200, 210, 220, 230, 240, 250, 260, 270, 280, 290, or 293 polynucleotides each is capable of detecting an expression product of a different gene shown in FIGS. 1-4.

In another embodiment, the invention provides an array consisting essentially of at least two antibody molecules or antigen binding fragments thereof, wherein each antibody molecule or antigen binding fragment thereof is capable of detecting an expressed gene product of a gene selected from the group consisting of genes shown in FIG. 1. In another embodiment, the invention provides an array consisting essentially of at least two antibody molecules or antigen binding fragments thereof, wherein each antibody molecule or antigen binding fragment thereof is capable of detecting an expressed gene product of a gene selected from the group consisting of genes shown in FIG. 2. In another embodiment, the invention provides an array consisting essentially of at least two antibody molecules or antigen binding fragments thereof, wherein each antibody molecule or antigen binding fragment thereof is capable of detecting an expressed gene product of a gene selected from the group consisting of genes shown in FIG. 3. In another embodiment, the invention provides an array consisting essentially of at least two antibody molecules or antigen binding fragments thereof, wherein each antibody molecule or antigen binding fragment thereof is capable of detecting an expressed gene product of a gene selected from the group consisting of genes shown in FIG. 4. In various embodiments, an antibody array in accordance with the invention consists essentially of at least any of 2, 3, 5, 10, 15, 20, 25, 30, 40, 50, 60, 70, 80, 90, 100, 110, 120, 130, 140, 150, 160, 170, 180, 190, 200, 210, 220, 230, 240, 250, 260, 270, 280, 290, or 293 antibodies or antigen binding fragments thereof each recognizing an expression product of a different gene shown in FIGS. 1-4.

### Kits

The invention also provides kits for methods of the present invention. For example, the invention provides kits containing a system for detecting gene expression, one or more polynucleotide sequences of the genes identified herein, one or more peptide products of the genes identified herein, and/or one or more antibodies that recognize polypeptide expression products of the differentially regulated genes described herein. A kit may contain diagnostic nucleotide probe sets, oligonucleotide or antibody microarrays, or reagents for performing an assay, packaged in a suitable container. The kit may further comprise one or more additional reagents, e.g., substrates, labels, primers, reagents for labeling expression products, tubes and/or other accessories, reagents for collecting tissue samples, buffers, hybridization chambers, cover slips, etc., and may also contain a software package, e.g., for analyzing differential expression using statistical methods as described herein, and optionally a password and/or account number for assessing the compiled database. The kit optionally further comprises an instruction set or user manual detailing preferred methods of performing the methods of the invention, and/or a reference to a site on the Internet where such instructions may be obtained.

The example provided below is to illustrate, but not to limit, the invention.

### EXAMPLE

### Preparation of Samples for Gene Expression Profiling

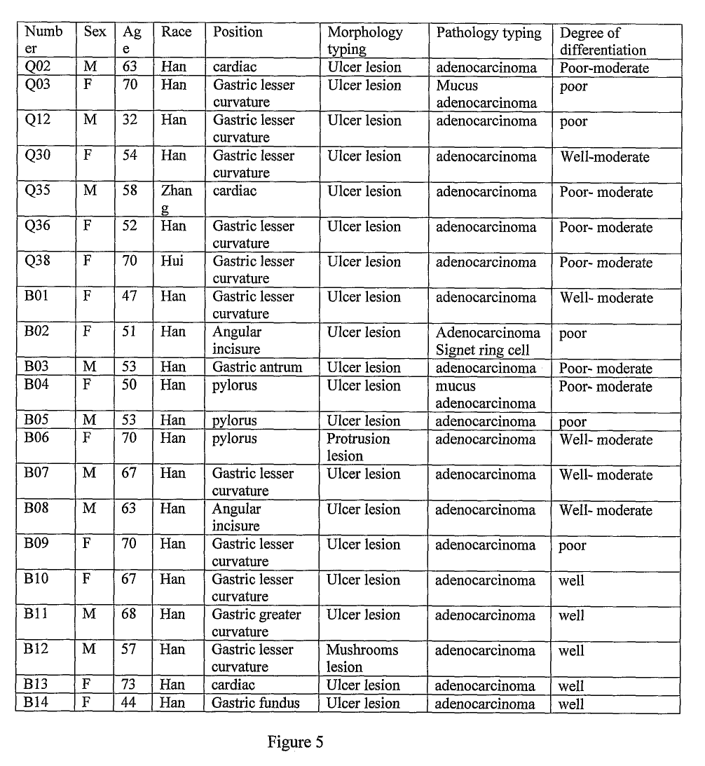

Intestinal gastric tissue samples from 21 cancer patients (see FIG. 5) were obtained from Tissue Bank of Peking University School of Oncology. Freshly excised cancer tissues and the normal tissues adjacent to the cancer tissues were placed in liquid nitrogen within 30 minutes after cessation of the blood supply. The tissues were obtained with consent of the patients, and the classifications of the tissues were confirmed by pathologists.

Intestinal gastric cancer tissues at various differentiation stages (such as highly differentiated and poorly differentiated) were analyzed. Generally, highly differentiated intestinal gastric cancer tissues (or gastric tissues of high level of differentiation) have obvious vascular structures with visible base membranes. The shapes and sizes of the vascular structures are relatively regular, and the cancer cells are typically in the shape of elongated columns or cubical structures with some visible abnormalities. Cell nucleuses are typically well-stained, some of which have migrated upwards. Poorly differentiated intestinal gastric cancer tissues (or gastric cancer tissues of low level of differentiation) are generally characterized by cells clusters or stripes or scattered single cells with obvious abnormalities. No obvious vascular structure can be observed, except that certain regions may show a tendency of forming a vascular structure. Generally, when compared to poorly differentiated gastric cancer tissues, highly differentiated gastric cancer tissues are less deteriorated and are relatively easier for prognosis.

Gastric mucosa tissue samples from normal people (specifically, from patients diagnosed of gentle chronic superficial gastritis) were obtained from Peking University Hospital. The samples were obtained with the consent of patients, and the classifications of the tissues were confirmed by pathologists.

Total mRNAs were extracted from the tissue samples with the TRIZOL reagent (Invitrogen, Gaithersbrug, Md., USA). The mRNAs were concentrated by isopropanol precipitation and further purified with an RNeasy mini kit (Qiagen, Valencia, Calif.) according to the manufacturer's instructions. The quality of the RNAs was assessed by formaldehyde agarose gel electrophoresis and was quantitated spectrophotometrically.

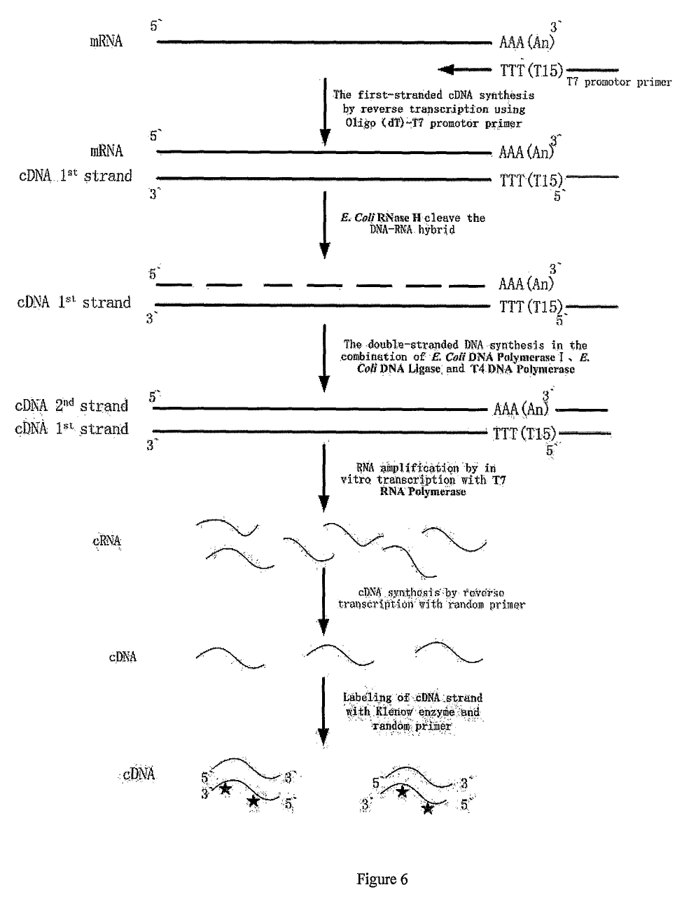

The mRNA samples were converted to cDNAs and labeled by fluorescent dye through Eberwine's linear RNA amplification method and subsequent enzymatic reaction. See FIG. 6. Specifically, double-stranded cDNA containing T7 RNA polymerase promoter sequence (5′-AAACG ACGGC CAGTG AATTG TAATA CGACT CACTA TAGGC GC-3′) was synthesized with 10 μg of total RNA using cDNA synthesis System Kit according to the protocol recommended by manufacturer (TaKaRa, Dalian, China). A T7-OligodT primer A T7-OligodT primer (5′-AAACG ACGGC CAGTG AATTG TAATA CGACT CACTA TAGGC GC TT TTT TTT TTT TTT TTTV-3′) was in replacement of poly T primer provided in the kit.

After completion of double stranded cDNA synthesis, the cDNA was purified with PCR Purification Kit (Qiagen), and eluted with 60 μl elution buffer. Half of the eluted double-strand cDNA product was vacuum-concentrated to 8 μl and subject to in vitro transcription reaction in 20 μL of reaction system using T7 RiboMAX Express Large Scale RNA Production System (Promega, Madison, Wis.). Reaction was allowed to continue for 3 hours at 37° C. and the amplified RNA (aRNA) was purified with RNeasy Mini kit (Qiagen).

cDNA labeling was achieved by using the Klenow enzyme following reverse transcription. Specifically, 1 μg aRNA was mixed with 2 μg of random primer (9mer), denatured at 70° C. for 5 min and cooled on ice. Then 4 μl of first strand buffer, 2 μl of 0.1M DTT, 1 μl 10 mM dNTP, and 1.5 μl SuperScript II (Invitrogen) were added. Tubes were incubated at 25° C. for 10 min and then at 42° C. for 60 min. The products were purified using a PCR purification kit (Qiagen) and vacuum-concentrated to 10 μl. cDNA was mixed with 2 μg random nonamer, heated to 95° C. for 3 min and snap cooled on ice. 10× buffer, dNTP and Cy5-dCTP or Cy3-dCTP (Amersham Pharmacia Biotech, Piscataway, N.J.) were added at final concentration of 120 μM each dATP, dGTP, dTTP, and 60 μM dCTP and 40 μM Cy-dye respectively. Klenow enzyme (1 μl, Takara) was then added and reaction was performed at 37° C. for 60 min. The labeled DNA was purified with a PCR purification kit (Qiagen), resuspended in Elution buffer and check O.D. Labeled control and test samples were quantitatively adjusted based on the efficiency of Cy-dye incorporation and mixed into 30 μl hybridization solution (3×SSC, 0.2% SDS, 25% formamide and 5× Denhart's).

### Preparation of DNA Microarrays

The human genome-wide long oligonucleotide microarray chips were constructed in Capital BioChip Corporation (Beijing, China). The oligonucleotides used for construction of the microarray chip (Human Genome Oligo Set Version 2.0) were obtained from Qiagen (Valencia, USA). The human Genome Oligo Set contains about 22000 DNA oligonucleotides with an average length of about 70 base. In addition to internal controls provided by the manufacturer, three Arabidopsis gene fragments of 70 bases were added as external controls. All nucleotides were dissolved in 50% DMSO to a final concentration of 40 μM and printed on in-house manufactured amino silaned glass slides. Arrays were fabricated by using an OmniGrid™ microarrayer (Genomic Instrumentation Services, Inc., San Carlos, Calif.). After printing, the slides were baked for one hour at 80° C. and stored dry at room temperature until use.

### Hybridization of the Sample to the DNA Microarray

Prior to hybridization, the slides prepared as above were rehydrated over 65° C. water for 10 seconds, snap dried on a 100° C. heating block for 5 seconds and UV cross-linked at 250 mJ/cm2. The unimmobilized oligonucleotides were washed off with 0.5% SDS for 15 minutes at room temperature and SDS was removed by dipping the slides in anhydrous ethanol for 30 seconds. The slides were spin-dried at 1000 rpm for 2 minutes.

DNA in hybridization solution prepared as above was denatured at 95° C. for 3 min prior loading on a microarray. The array was hybridized at 42° C. overnight and washed with two consecutive washing solutions (0.2% SDS, 2×SSC at 42° C. for 5 min, and 0.2% SSC for 5 min at room temperature). The washed slides were then snap-dried and ready for scanning.

### Imaging and Data Analysis

The arrays were scanned with a ScanArray Express Scanner (Parckard Bioscience, Kanata, OT, USA), and the images obtained were analyzed with GenePix Pro 4.0 (Axon Instruments, Foster City, Calif., USA). The signals were digitalized and normalized by the LOWESS method. Genes were determined to be differentially expressed if the compared signals were at least two times different.

The data were analyzed using data analysis computer software packages generally recognized in the field as well as software developed in house. Specifically, the identification of differentially expressed genes was carried out by using the BRB ArrayTools and SAM, chip data analysis software 1.0, and Cluster 3.0. Functional analyses were carried out by using GO database. Nucleic Acids Research, 2004, 32:D258-261. Identifications of chromosomal locations of genes were carried out by using BRB ArrayTools and MACAT. A database of genes differentially expressed in gastric cancer tissues is thereby established.

### Gene Differentially Expressed in Gastric Cancer Tissues

The gene expression profiles of the 21 intestinal gastric cancer tissue samples were each compared with those of the corresponding adjacent tissue samples. Differential expression of 102 genes was observed in more than 80% samples. See FIG. 1. Among the 102 genes, 31 genes (i.e., genes 72-102) were overexpressed in cancer tissue samples and 71 genes (i.e., genes 1-71) were underexpressed in cancer tissue samples.

The expression profiles of cancer tissue samples and corresponding adjacent tissue samples were further compared with those of gastric mucosa tissue samples obtained from normal people to identify genes that were differentially expressed. For genes that did not show apparent differential expression between cancer tissue samples and corresponding adjacent tissue samples, we further compared their expressions in cancer tissue samples (and corresponding adjacent tissue samples) with their expression in gastric mucosa tissue samples obtained from normal people. We found 84 genes with differential expression in more than 80% of the samples. See FIG. 2. Among these 84 genes, 31 genes (i.e., genes 54-84) were overexpressed in cancer tissues and corresponding adjacent tissues and 53 genes (i.e., genes 1-53) were underexpressed (as compared to gastric mucosa tissue samples obtained from normal people).

We have also identified genes that are differentially expressed in highly or poorly differentiated cancer tissues. Specifically, when expression profiles of highly differentiated intestinal gastric cancer tissue samples were compared with the corresponding normal adjacent tissues (see FIG. 3), we found 16 genes (i.e., genes 1-16) that were overexpressed in cancer tissues and 39 genes (i.e., genes 17-55) that were underexpressed. When expression profiles of poorly differentiated intestinal gastric cancer tissue samples were compared with the corresponding normal adjacent tissues (FIG. 4), we found 28 genes (i.e., genes 1-28) that were overexpressed in cancer tissue and 18 genes (i.e., genes 29-46) that were underexpressed. These genes can be used in determining the extent of differentiation of intestinal gastric cancer tissue samples, and are particularly useful in classification or diagnosis of intestinal gastric cancer, as well as serving as a basis for individualized treatment.

The differential expressions were further confirmed by semi-quantitative RT/PCR and Western Blot. Furthermore, gene and protein expression changes were also identified by high throughput tissue microarray in combination with in situ hybridization and immunohistochemistry (data not shown). FIG. 7 provides exemplary data on differential expression of the THY1 gene (GB accession: AK057865) based on RT-PCR and tissue array. The molecular data, in combination with clinical information, provide a good tool in gastric cancer diagnosis and prognosis.

What is claimed is:

1. A method for diagnosing intestinal gastric cancer in a mammal, comprising:
(a) detecting levels of expression of at least two genes shown in FIG. 1 in an intestinal gastric tissue of the mammal, wherein the tissue is suspected of being cancerous;
(b) detecting levels of expression of the genes in an intestinal gastric tissue adjacent to the tissue suspected of being cancerous of the mammal; and
(c) comparing the levels of expressions of the genes in the tissue suspected of being cancerous to the levels of expression in the adjacent tissue; wherein substantial variance of the levels of expression of the genes between the tissue suspected of being cancerous and the adjacent tissue is indicative of presence of intestinal gastric cancer in the mammal.

2. The method of claim 1, wherein the levels of expression of all the genes in FIG. 1 are detected, wherein substantially increased expression of at least 18 of genes 72-102 in FIG. 1 and substantially decreased expression of at least 42 of genes 1-71 in FIG. 1 in the tissue suspected of being cancerous as compared to the adjacent tissue are indicative of presence of intestinal gastric cancer in the mammal.

3. The method of claim 1, wherein the levels of expression of all the genes in FIG. 1 are detected, wherein substantially increased expression of genes 72-102 in FIG. 1 and substantially decreased expression of genes 1-71 in FIG. 1 in the tissue suspected of being cancerous as compared to the adjacent tissue are indicative of presence of intestinal gastric cancer in the mammal.

4. The method of claim 1, wherein the levels of expression of the genes are detected by detecting the levels of mRNA encoded by the genes.

5. The method of claim 1, wherein the levels of expression of the genes are detected by detecting the levels of the protein encoded by the genes.

6. A system for diagnosing intestinal gastric cancer in a mammal, consisting essentially of a plurality of isolated polynucleotide molecules, wherein each isolated polynucleotide molecule is capable of detecting expression of a gene selected from the group consisting of at least 61 genes shown in FIG. 1, whereby differential expression of said genes can be detected.

7. The system of claim 6, wherein the isolated polynucleotide molecules are selected from the group consisting of DNA, RNA, and PNA.

8. A kit comprising the system of claim 6.

9. The kit of claim 8, wherein the isolated polynucleotide molecules are immobilized on an array.

10. A method for diagnosing intestinal gastric cancer in a mammal, comprising:
(a) detecting levels of expression of at least two genes shown in FIG. 2 in an intestinal gastric tissue suspected of being cancerous of the mammal;
(b) detecting levels of expression of the genes in an intestinal gastric tissue adjacent to the gastric tissue suspected of being cancerous;
(c) comparing the levels of expression of the genes in the gastric tissue suspected of being cancerous to levels of expression of the genes in a normal intestinal gastric mucosa tissue of the same mammal species;
(d) comparing the levels of expression of the genes in the adjacent gastric tissue to levels of expression of the genes in a normal intestinal gastric mucosa tissue of the same mammal species; and
(e) comparing the levels of expression of the genes in the gastric tissue suspected of being cancerous to the levels of expression of the genes in the adjacent gastric tissue;
wherein substantial variance of the levels of expression of the genes shown in FIG. 2 between the gastric tissue suspected of being cancerous and the normal gastric mucosa tissue and between the adjacent gastric tissue and the normal gastric mucosa tissue, and no substantial variance of the levels of expression of the genes shown in FIG. 2 between the gastric tissue suspected of being cancerous and the adjacent gastric tissue is indicative of presence of intestinal gastric cancer in the mammal.

11. The method of claim 10, wherein the levels of expression of all the genes in FIG. 2 is detected, wherein substantially increased expression of at least 18 of genes 54-84 in FIG. 2 and substantially decreased expression of at least 31 of genes 1-53 in FIG. 2 in the gastric tissue suspected of being cancerous and the adjacent tissue as compared to the normal gastric mucosa tissue are indicative of presence of intestinal gastric cancer in the mammal.

12. The method of claim 10, wherein the levels of expression of all the genes in FIG. 2 is detected, wherein substantially increased expression of genes 54-84 in FIG. 2 and substantially decreased expression of genes 1-53 in FIG. 2 in the gastric tissue suspected of being cancerous and the adjacent tissue as compared to the normal gastric mucosa tissue are indicative of presence of intestinal gastric cancer in the mammal.

13. The method of claim 10, wherein the levels of expression of the genes is detected by detecting the levels of mRNA encoded by the genes.

14. The method of claim 10, wherein the levels of expression of the genes is detected by detecting the levels of the protein encoded by the genes.

15. A system for diagnosing intestinal gastric cancer in a mammal, consisting essentially of a plurality of isolated polynucleotide molecules, wherein each isolated polynucleotide molecule is capable of detecting expression of a gene selected from the group consisting of at least 50 genes shown in FIG. 2, whereby differential expression of said genes can be detected.

16. The system of claim 15, wherein the isolated polynucleotide molecules are selected from the group consisting of DNA, RNA, and PNA.

17. A kit comprising the system of claim 15.

18. The kit of claim 17, wherein the isolated polynucleotide molecules are immobilized on an array.

19. A method for assessing levels of differentiation of intestinal gastric cancer in a mammal, comprising:
(a) detecting levels of expression of at least two genes shown in FIG. 3 in an intestinal gastric cancer tissue of the mammal;
(b) detecting levels of expression of the genes in an intestinal gastric tissue adjacent to the cancer tissue; and
(c) comparing the levels of expression of the genes in the cancer tissue to the levels of expression of the genes in the adjacent tissue;
wherein substantial variance of the levels of expression of the genes between the cancer tissue and the adjacent tissue is indicative of high level of differentiation of the intestinal gastric cancer in the mammal.

20. The method of claim 19, wherein the levels of expression of all the genes in FIG. 3 is detected, wherein substantially increased expression of at least 9 of genes 1-16 in FIG. 3 and substantially decreased expression of at least 23 of genes 17-55 in FIG. 3 in the cancer tissue as compared to the adjacent tissue are indicative of high level of differentiation of the intestinal gastric cancer in the mammal.

21. The method of claim 19, wherein the levels of expression of all the genes in FIG. 3 is detected, wherein substantially increased expression of genes 1-16 in FIG. 3 and substantially decreased expression of genes 17-55 in FIG. 3 in the cancer tissue as compared to the adjacent tissue are indicative of high level of differentiation of the intestinal gastric cancer in the mammal.

22. The method of claim 19, wherein the levels of expression of the gene is detected by detecting the levels of mRNA encoded by the gene.

23. The method of claim 19, wherein the levels of expression of the gene is detected by detecting the levels of the protein encoded by the gene.

24. A system for assessing levels of differentiation of intestinal gastric cancer in a mammal, consisting essentially of a plurality of isolated polynucleotide molecules, wherein each isolated polynucleotide molecule is capable of detecting expression of a gene selected from the group consisting of at least 33 genes shown in FIG. 3, whereby differential expression of said genes can be detected.

25. The system of claim 24, wherein the isolated polynucleotides are selected from the group consisting of DNA, RNA, and PNA.

26. A kit comprising the system of claim 24.

27. The kit of claim 26, wherein the isolated polynucleotides are immobilized on an array.

28. A method for assessing levels of differentiation of intestinal gastric cancer in a mammal, comprising:
(a) detecting levels of expression of at least two genes shown in FIG. 4 in an intestinal gastric cancer tissue of the mammal;
(b) detecting levels of expression of the genes in an intestinal gastric tissue adjacent to the cancer tissue; and
(c) comparing the levels of expression of the genes in the cancer tissue to the levels of expression in the adjacent tissue;
wherein substantial variance of the levels of expression of the genes between the cancer tissue and the adjacent tissue is indicative of low level of differentiation of the intestinal gastric cancer in the mammal.

29. The method of claim 28, wherein the levels of expression of all the genes in FIG. 4 is detected, wherein substantially increased expression of at least 16 of genes 1-28 in FIG. 4 and substantially decreased expression of at least 10 of genes 29-46 in FIG. 4 in the cancer tissue as compared to the adjacent tissue are indicative of low level of differentiation of the intestinal gastric cancer in the mammal.

30. The method of claim 28, wherein the levels of expression of all the genes in FIG. 4 is detected, wherein substantially increased expression of genes 1-28 in FIG. 4 and substantially decreased expression of genes 29-46 in FIG. 4 in the cancer tissue as compared to the adjacent tissue are indicative of high level of differentiation of the intestinal gastric cancer in the mammal.

31. The method of claim 28, wherein the levels of expression of the genes is detected by detecting the levels of mRNA encoded by the genes.

32. The method of claim 28, wherein the levels of expression of the genes is detected by detecting the levels of the protein encoded by the genes.

33. A system for assessing levels of differentiation of intestinal gastric cancer in a mammal, consisting essentially of a plurality of isolated polynucleotide molecules, wherein each isolated polynucleotide molecule is capable of detecting expression of a gene selected from the group consisting of at least 27 genes shown in FIG. 4, whereby differential expression of said genes can be detected.

34. The system of claim 33, wherein the isolated polynucleotides are selected from the group consisting of DNA, RNA, and PNA.

35. A kit comprising the system of claim 33.

36. The kit of claim 35, wherein the isolated polynucleotide molecules are immobilized on an array.
